(19) United States
(12) Patent Application Publication
(10) Pub. No.: US 2017/0315348 A1
(43) Pub. Date: Nov. 2, 2017

(54) DISPLAY DEVICE AND DISPLAY CONTROL METHOD

(71) Applicant: SONY CORPORATION, TOKYO (JP)

(72) Inventor: MASATAKE HAYASHI, KANAGAWA (JP)

(21) Appl. No.: 15/522,084

(22) PCT Filed: Oct. 6, 2015
(86) PCT No.: PCT/JP2015/078276

§ 371 Date: Apr. 26, 2017

(30) Foreign Application Priority Data
Nov. 7, 2014 (JP) 2014-227279

(51) Int. Cl. G02B 27/00; G02F 1/1335; A61B 3/103; G09G 3/36; G02B 3/00

(52) U.S. Cl. CPC G02B 27/0093; G09G 3/36; G02B 3/0037; A61B 3/103; G02F 1/133526; G09G 2320/08; G09G 2354/00

(57) ABSTRACT

Provided is a display device (10) including a plurality of light emission points (125). A region group (209) including a plurality of regions (207) which are set on a plane (205) including a pupil of a user is irradiated with light emitted from each of the plurality of light emission points, each of the plurality of light emission points causes light corresponding to a combination of the light emission point and the region to be incident on each of the regions, a number of the regions set on the pupil of the user is two or more, and a size of each of the regions is smaller than 0.6 (mm), and the display device is capable of providing display that is more favorable to a user with presbyopia or myopia.

### TECHNICAL FIELD

The present disclosure relates to a display device and a display control method.

### BACKGROUND ART

For display devices, increasing an amount of information to be displayed on a screen is an important mission. In view of this, in recent years, display devices capable of performing display with higher resolution such as, for example, 4K television, have been developed. Particularly, in a device having a relatively small display screen size such as a mobile device, higher-definition display is required to display more information on a small screen.

However, in addition to increasing the amount of information to be displayed on the display device, high visibility is also required. Even if higher-resolution display is performed, a degree of resolution to which display can be determined depending on the visual acuity of an observer (user). In particular, it is assumed that it is difficult for elderly users to visually recognize high-resolution displays due to presbyopia with aging.

Generally, as countermeasures against presbyopia, optical compensation instruments such as presbyopic glasses are used. However, because far visual acuity is degraded while presbyopic glasses are worn, attachment/detachment is necessary in accordance with a situation. Also, it is necessary to carry a tool for storing presbyopic glasses such as an eyeglass case in accordance with the necessity of attachment/detachment. For example, it is necessary for a user with presbyopia who uses a mobile device to carry a tool having a volume equal to or larger than that of the mobile device, so that portability, which is an advantage of the mobile device, is impaired, which feels annoying to many users. Furthermore, many users feel resistance to wearing presbyopic glasses themselves.

Therefore, in a display device, particularly, a display device having a relatively small display screen mounted on a mobile device, technology in which the display device itself improves visibility for a user without using additional devices such as presbyopic glasses is desired. For example, in Patent Literature 1, technology in which a plurality of lenses are arranged so that images of pixel groups are overlapped and projected in a display device including the plurality of lenses and a plurality of light emission point (pixel) groups and the projected images from the plurality of lenses are formed on the retina of a user by causing an overlap of pixels in the pixel groups projected and overlapped by the lenses to be incident on a user's pupil is disclosed. In the technology described in Patent Literature 1, an image with a deep focal depth is formed on the retina by adjusting a projection size of light from a pixel on the pupil to a size smaller than a pupil diameter and a user with presbyopia can also obtain an in-focus image.

### CITATION LIST

### Patent Literature

Patent Literature 1: JP 2011-191595A

### DISCLOSURE OF INVENTION

### Technical Problem

However, in the technology described in Patent Literature 1, in principle, when two or more light beams corresponding to the overlap of pixels in the pixel groups projected and overlapped by the lenses are incident on the pupil, the image on the retina will be blurred. Accordingly, in the technology described in Patent Literature 1, adjustment is performed so that an interval between light beams corresponding to the overlap of the pixels on the pupil (that is, projected images on the pupil of light from the pixels) is set to be larger than the pupil diameter and a plurality of light beams are not incident simultaneously.

However, in this configuration, when a position of the pupil has moved with respect to the lens, there is a moment when the light beam is not incident on the pupil. While the light beam is not incident on the pupil, no image is visually recognized by the user and the user can observe an invisible region such as a black frame. Because the invisible region is periodically generated every time the pupil moves by about the pupil diameter, it cannot be said that comfortable display is provided for the user.

Therefore, the present disclosure provides a novel and improved display device and display control method capable of providing display that is more favorable to a user.

### Solution to Problem

According to the present disclosure, there is provided a display device including: a plurality of light emission points. A region group including a plurality of regions which are set on a plane including a pupil of a user is irradiated with light emitted from each of the plurality of light emission points, each of the plurality of light emission points causes light corresponding to a combination of the light emission point and the region to be incident on each of the regions, and a number of the regions set on the pupil of the user is two or more, and a size of each of the regions is smaller than 0.6 (mm).

According to the present disclosure, there is provided a display control method including: irradiating a region group including a plurality of regions which are set on a plane including a pupil of a user with light emitted from each of a plurality of light emission points, and also causing light corresponding to a combination of the light emission point and the region to be incident on each of the regions from each of the plurality of light emission points. A number of the regions set on the pupil of the user is two or more, and a size of each of the regions is smaller than 0.6 (mm).

According to the present disclosure, a plurality of regions each having a size smaller than 0.6 (mm) are set on a plane including a pupil of a user so that the number of the regions located on the pupil of the user is two or more, and an irradiation state of light with respect to each of the regions is controlled. With the control on the irradiation state of the light with respect to each of the plurality of regions on the pupil, display for compensating for visual acuity of the user can be performed, for example, a virtual image located a predetermined distance away from the user can be displayed to the user. Further, according to this configuration, since light beams which are appropriately controlled are incident on each of the plurality of regions on the pupil, the invisible region as in the technology described in Patent Literature 1 is not generated even in the case where a viewpoint of the user has moved.

### Advantageous Effects of Invention

According to the present disclosure as described above, display that is more favorable to a user can be provided. Note that the effects described above are not necessarily limitative. With or in the place of the above effects, there may be achieved any one of the effects described in this specification or other effects that may be grasped from this specification.

### BRIEF DESCRIPTION OF DRAWINGS

FIG. 1 is a graph illustrating an example of relationships between limit resolution and visual acuity and a viewing distance.

FIG. 2 is a graph illustrating an example of relationships between a limit resolution of a user with emmetropia and an age and a viewing distance.

FIG. 3 is a graph illustrating an example of relationships between a limit resolution of a user with myopia and an age and a viewing distance.

FIG. 4 is an explanatory diagram illustrating a concept for assigning depth information to two-dimensional picture information.

FIG. 5 is a diagram illustrating an example of a configuration of a light-ray reproduction display device.

FIG. 6 is a diagram illustrating an example of a configuration of a display device that displays a general two-dimensional picture.

FIG. 7 is a schematic diagram illustrating a state in which a user's focus is aligned with a display surface in a general two-dimensional display device.

FIG. 8 is a schematic diagram illustrating a state in which the user's focus is not aligned with the display surface in the general two-dimensional display device.

FIG. 9 is a schematic diagram illustrating a relationship between a virtual image surface in the light-ray reproduction display device and an image formation surface on a retina of the user.

FIG. 10 is a diagram illustrating an example of a configuration of a display device according to a present embodiment.

FIG. 11 is a diagram illustrating a light ray emitted from a microlens in a normal mode.

FIG. 12 is a diagram illustrating a specific display example of a pixel array in the normal mode.

FIG. 13 is a diagram illustrating a positional relationship between a virtual image surface and a display surface of a microlens array in the normal mode.

FIG. 14 is a diagram illustrating a light ray emitted from a microlens in a visual acuity compensation mode.

FIG. 15 is a diagram illustrating a specific display example of a pixel array in the visual acuity compensation mode.

FIG. 16 is a diagram illustrating a positional relationship between a virtual image surface and a display surface of a microlens array in the visual acuity compensation mode.

FIG. 17 is a diagram illustrating a relationship between a pupil diameter of a pupil of a user and a size of a sampling region.

FIG. 18 is a diagram illustrating a relationship between λ and PD when an iteration cycle λ satisfies Equation (3).

FIG. 19 is a diagram illustrating a relationship between λ and PD when an iteration cycle λ satisfies Equation (4).

FIG. 20 is a diagram illustrating an influence of the relationship between an iteration cycle λ and PD on a size of a continuous display region.

FIG. 21 is a flowchart illustrating an example of a processing procedure of a display control method according to the present embodiment.

FIG. 22 is a diagram illustrating an example of a configuration in which a display device according to the present embodiment is applied to a wearable device.

FIG. 23 is a diagram illustrating an example of a configuration in which a display device according to the present embodiment is applied to another mobile device.

FIG. 24 is a diagram illustrating an example of a general electronic loupe device.

FIG. 25 is a schematic diagram illustrating a state of a decrease of a pixel size dp due to a first shielding plate having a rectangular opening (aperture).

FIG. 26 is a schematic diagram illustrating a state of a decrease of a pixel size dp due to a first shielding plate having a circular opening (aperture).

FIG. 27 is a diagram illustrating an example of a configuration in which the first shielding plate is provided between a backlight and a liquid crystal layer.

FIG. 28 is a diagram illustrating an example of a configuration of a display device according to a modified example in which dynamic control of an irradiation state in accordance with pupil position detection is performed.

### MODE(S) FOR CARRYING OUT THE INVENTION

Hereinafter, (a) preferred embodiment(s) of the present disclosure will be described in detail with reference to the appended drawings. In this specification and the appended drawings, structural elements that have substantially the same function and structure are denoted with the same reference numerals, and iterated explanation of these structural elements is omitted.

Also, the description will be given in the following order.

1. Background of present disclosure
2. Basic principle of present embodiment
3. Display device according to present embodiment
3-1. Device configuration
3-2. Driving example
3-2-1. Normal mode
3-2-2. Visual acuity compensation mode
3-3. Detailed design
3-3-1. Sampling region
3-3-2. Iteration cycle of irradiation state of sampling region
4. Display control method
5. Application examples
5-1. Application to wearable device
5-2. Application to other mobile devices
5-3. Application to electronic loupe device
6. Modified example
6-1. Decrease of pixel size in accordance with aperture
6-2. Example of configuration of light emission point other than microlens
6-3. Dynamic control of irradiation state in accordance with pupil position detection
6-4. Modified example in which pixel array is implemented by printing material

### 7. Supplement

### 1. Background of Present Disclosure

First, prior to describing a preferred embodiment of the present disclosure, a background that the present inventors have conceived for the present disclosure will be described.

As described above, in recent years, display devices capable of performing display with higher resolution have been developed. Particularly, in a device having a relatively small display screen size such as a mobile device, higher-definition display is required to display more information on a small screen.

However, the resolution capable of being distinguished by a user depends on the visual acuity of the user. Accordingly, even when a resolution beyond a limit of the visual acuity of the user is pursued, an advantage is not necessarily given to the user.

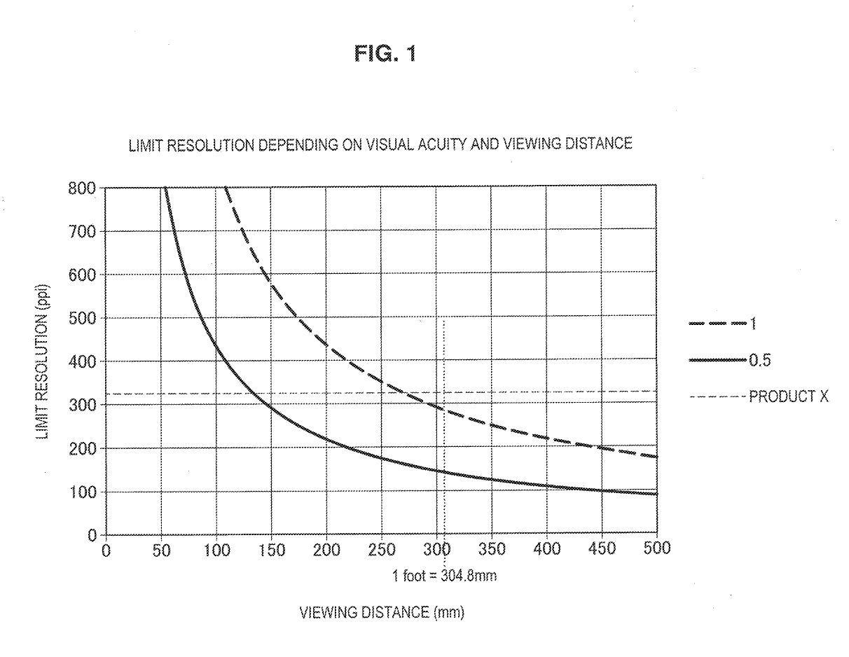

Relationships between the resolution (limit resolution) capable of being distinguished by a user and visual acuity and a viewing distance (a distance between the display surface of the display device and the pupil of the user) are illustrated in FIG. 1. FIG. 1 is a graph illustrating the relationships between the limit resolution and the visual acuity and the viewing distance. In FIG. 1, the viewing distance (mm) is taken on the horizontal axis, the limit resolution (ppi: pixels per inch) is taken on the vertical axis, and a relationship between the two is plotted. Also, the visual acuity is taken as a parameter and the relationship between the viewing distance and the limit resolution is plotted for a case in which the visual acuity is 1.0 and a case in which the visual acuity is 0.5.

Referring to FIG. 1, it can be seen that as the viewing distance increases, that is, as the distance between the display surface and the pupil increases, the limit resolution decreases. Also, it can be seen that the lower the visual acuity, the lower the resolution limit.

Here, the resolution of a product X that is generally distributed is about 320 (ppi) (indicated by a broken line in FIG. 1). From FIG. 1, it can be seen that the resolution of the product X is set to be slightly larger than the limit resolution at the viewing distance 1 (foot) (=304.8 (mm)) of a user whose visual acuity is 1.0. That is, in the product X, the resolution effectively functions in the sense that pixels cannot be recognized for a user having a visual acuity of 1.0 viewing the display surface from the distance of 1 (foot).

On the other hand, visual acuity differs depending on a user. Some users have myopia where visual acuity is degraded at a long distance, and others have presbyopia where visual acuity is degraded at a short distance due to aging. When considering the relationship between the limit resolution and the resolution of the display surface, it is also necessary to consider such a change in the visual acuity of the user depending on the viewing distance. In the example illustrated in FIG. 1, the limit resolution at the viewing distance 1 (foot) of a user whose visual acuity is 0.5 is about 150 (ppi), and only about half of the resolution of the product X can be distinguished at the same viewing distance of 1 (foot) of the user.

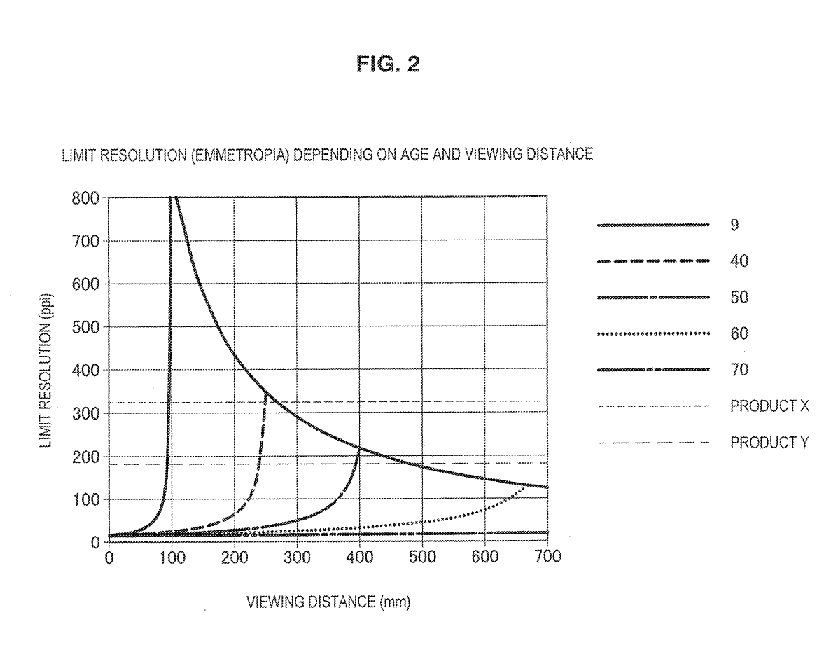

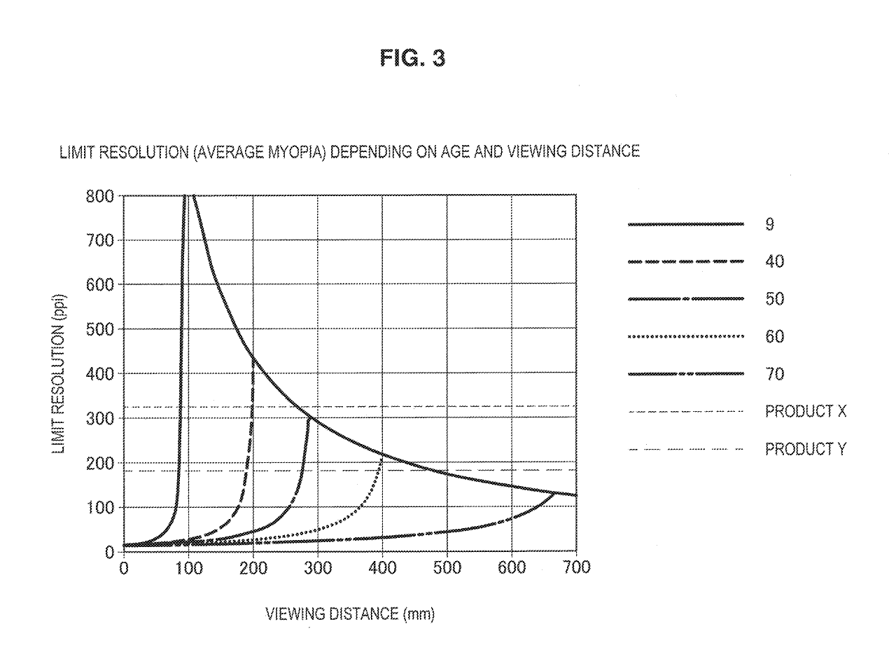

A user with presbyopia is considered with reference to FIGS. 2 and 3. FIG. 2 illustrates an example in which relationships between a limit resolution of a user with emmetropia with a far-field visual acuity of 1.0 and an age and a viewing distance are approximated. In FIG. 2, the viewing distance (mm) is taken on the horizontal axis, the limit resolution (ppi) of a user with general emmetropia is taken on the vertical axis, and a relationship between the two is plotted. Also, when the age is taken as a parameter and the age is 9 years old, 40 years old, 50 years old, 60 years old, and 70 years old, the relationship between the viewing distance and the limit resolution is plotted.

Also, an example in which relationships between the limit resolution of a user having standard myopia to the extent that a lens of −1.0 (diopter) is appropriate for far-field vision and an age and a viewing distance are approximated is illustrated in FIG. 3. FIG. 3 is a graph illustrating an example in which relationships between a limit resolution of a user with myopia and an age and a viewing distance are approximated. In FIG. 3, the viewing distance (mm) is taken on the horizontal axis, the limit resolution (ppi) of a general myopia user is taken on the vertical axis, and the relationship between the two is plotted. Also, when the age is taken as a parameter and the age is 9 years old, 40 years old, 50 years old, 60 years old, and 70 years old, a relationship between the viewing distance and the limit resolution is plotted.

Referring to FIGS. 2 and 3, it can be seen that the limiting resolution decreases with the age with respect to both the user with emmetropia and the user with myopia. This is due to presbyopia progressing with aging. In FIGS. 2 and 3, together with the resolution of the product X illustrated in FIG. 1, the resolution of another product Y is also shown. The resolution of the product Y is about 180 (ppi) (indicated by a different type of broken line from that for the product X in FIGS. 2 and 3).

From FIG. 2, it can be seen that the resolution of the product X cannot be substantially distinguished by a user of 40 years old or more having emmetropia. Also, referring to FIG. 3, it can be seen that, although the decrease of the limit resolution in accordance with aging is gentle for a user with myopia compared to a user with emmetropia, the resolution of the product X cannot be substantially distinguished for users of 50 years old or more.

Here, referring to FIGS. 2 and 3, if the viewing distance is around 250 (mm), for example, for a user of 40 years old, there is a possibility of their limit resolution exceeding the resolution of product X and it being possible to distinguish the resolution of product X. However, the range of the viewing distance where the limit resolution exceeds the resolution of product X is extremely limited. The limit resolution decreases due to presbyopia when the viewing distance becomes closer and the limit resolution decreases due to a limit of visual acuity in accordance with a distance to the display surface when the viewing distance becomes farther away. It is not desirable for the user to visually recognize the display surface in a state in which the viewing distance is always kept within the range in terms of comfortable use.

As described above, for a user with presbyopia of, for example, 40 years old or more, it is difficult to say that the resolution enhancement of about 300 (ppi) or more is meaningful from a viewpoint of the benefit to the user. However, despite the fact that the amount of information handled by users has increased in recent years, devices handled by users like mobile devices have tended to become miniaturized. Accordingly, it is an inevitable requirement to increase an information density in the display screen in, for example, mobile devices such as smart phones and wearable devices.

As a method of improving the visibility for the user, it is conceivable to decrease the density of the information on the display screen, such as increasing a character size of the display screen. However, this method is contrary to a demand for higher density of information. Also, if the density of the information on the display screen decreases, the amount of information given to the user on one screen decreases and the usability for the user also decreases. Alternatively, it is conceivable to increase the amount of information on one screen by increasing the size of the display screen itself, but, in that case, portability, which is an advantage of the mobile device, deteriorates.

While there is a demand to provide a high-resolution display screen having a larger information density amount for all users including the elderly as described above, there is a limit due to the user's visual acuity in the resolution capable of being distinguished by the user.

Here, as described above, in general, optical compensation instruments such as presbyopic glasses are widely used as a countermeasure against presbyopia. However, presbyopic glasses need to be attached and detached in accordance with the distance to an observation object. In accordance with this, it is necessary to carry tools for storing presbyopic glasses such as eyeglass cases. It is necessary for users using mobile devices to carry tools with a volume equal to or larger than that of the mobile device, which feels annoying to many users. Further, many users feel resistance to wearing presbyopic glasses themselves.

In view of the above circumstances, there has been a demand for technology capable of providing favorable visibility for a user in which high-resolution display is able to be distinguished without using additional instruments such as presbyopic glasses. The present inventors have conceived the following embodiments of the present disclosure as a result of diligently studying technology capable of providing favorable visibility for a user by devising the configuration of a display device without using additional instruments such as presbyopic glasses.

Hereinafter, one embodiment conceived by the present inventors as preferred embodiments of the present disclosure will be described.

### 2. Basic Principle of Present Embodiment

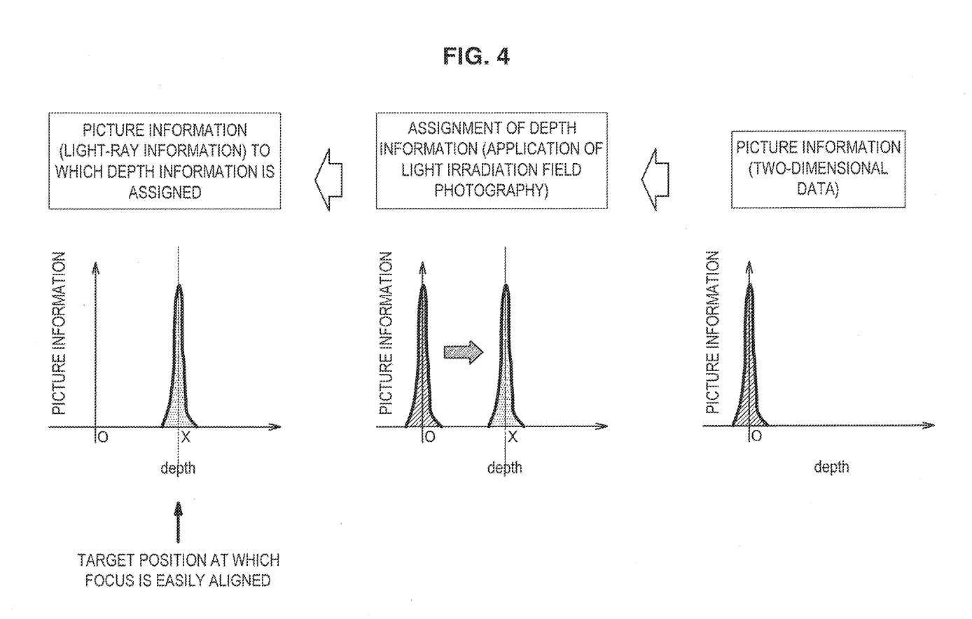

First, prior to describing a specific device configuration, the basic principle of the present embodiment will be described with reference to FIG. 4. FIG. 4 is an explanatory diagram illustrating a concept of assigning depth information to two-dimensional picture information.

As illustrated in the right diagram of FIG. 4, in a general display device, picture information is displayed as a two-dimensional picture on the display surface. Two-dimensional picture information can be said to be picture information without depth information.

Here, there is technology called irradiation field photography as photographic technology capable of obtaining pictures at various focal positions through calculation by acquiring information about both a position and a direction of light rays in a space of a subject without obtaining information about the intensity of light incident from each direction as in a normal photographing device when the subject is photographed. This technology can be implemented by performing a process of simulating a state of image formation within a camera through calculation on the basis of a light-ray state within the space (light field).

On the other hand, as technology for reproducing information of the light-ray state (light field) in a real space, technology called light-ray reproduction technology is also known. In the example illustrated in FIG. 4, the light-ray state in a case in which the display surface is present at the position X is first obtained through calculation, and the obtained light-ray state is reproduced by the light-ray reproduction technology, so that a real display surface is located at a position O, but it is possible to reproduce the light-ray state as if the display surface were located at a position X different from the position O (see the middle drawing in FIG. 4). Information of the light-ray state (light-ray information) can also be said to be three-dimensional picture information in which information about the position in a depth direction of a virtual display surface is assigned to two-dimensional picture information.

By reproducing a light-ray state as if the display surface were located at the position X in accordance with the light-ray information and irradiating the user's pupil with light in the irradiation state based on the light-ray state, the user visually recognizes an image on a virtual display surface (that is, a virtual image) located at the position X. If the position X is adjusted to a position in focus for, for example, a user with presbyopia, it is possible to provide an in-focus picture to the user.

As such a display device for reproducing a predetermined light-ray state on the basis of light-ray information, several light-ray reproduction type display devices are known. The light-ray reproduction type display device is configured so that light from each pixel can be controlled in accordance with an emission direction, and is widely used as, for example, a naked-eye 3D display device that provides 3D pictures by emitting light so that a picture taking into consideration binocular parallax on left and right eyes of the user is recognized.

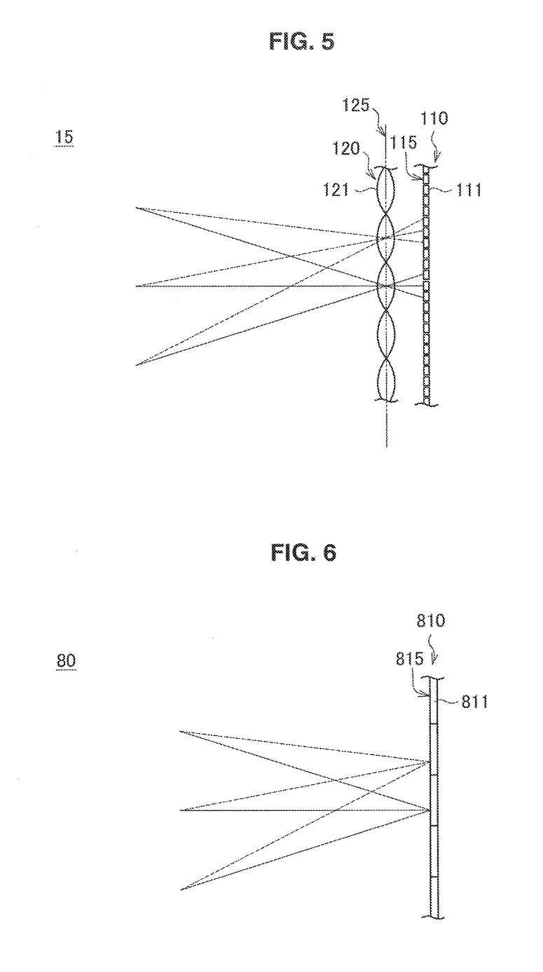

An example of the configuration of the light-ray reproduction type display device is illustrated in FIG. 5. FIG. 5 is a diagram illustrating an example of the configuration of the light-ray reproduction type display device. Also, for comparison, an example of a configuration of a display device that displays a general two-dimensional picture is illustrated in FIG. 6. FIG. 6 is a diagram illustrating an example of a configuration of a display device that displays a general two-dimensional picture.

Referring to FIG. 6, a display surface of a general display device 80 includes a pixel array 810 in which a plurality of pixels 811 are two-dimensionally arranged. In FIG. 6, for convenience, the pixel array 810 is illustrated as if the pixels 811 were arranged in one column, but, in reality, the pixels 811 are arranged also in the depth direction of the drawing sheet. The amount of light from each pixel 811 is not controlled depending on the emission direction, and a controlled amount of light is similarly emitted in any direction. The two-dimensional picture described with reference to the drawing on the right side of FIG. 4 indicates, for example, a two-dimensional picture displayed on the display surface 815 of the pixel array 810 illustrated in FIG. 6. Hereinafter, in order to distinguish it from the light-ray reproduction type display device, a display device 80 for displaying a two-dimensional picture (that is, picture information without depth information) as represented in FIG. 6 is also referred to as a two-dimensional display device 80.

Referring to FIG. 5, a light-ray reproduction type display device 15 includes a pixel array 110 in which a plurality of pixels 111 are two-dimensionally arranged and a microlens array 120 provided on a display surface 115 of the pixel array 110. In FIG. 5, for convenience, the pixel array 110 is illustrated as if the pixels 111 were arranged in one column, but the pixels 111 are also actually arranged in a depth direction of the drawing sheet. Likewise, also in the microlens array 120, the microlenses 121 are actually arranged in the depth direction of the drawing sheet. Because the light from each pixel 111 is emitted through the microlens 121, the lens surface 125 of the microlens array 120 becomes an apparent display surface 125 in the light-ray reproduction type display device 15.

A pitch of the microlenses 121 in the microlens array 120 is configured to be larger than the pitch of the pixels 111 in the pixel array 110. That is, a plurality of pixels 111 are located immediately below one microlens 121. Accordingly, light from the plurality of pixels 111 is incident on one microlens 121, and is emitted with directivity. Consequently, by appropriately controlling the driving of each pixel 111, it is possible to adjust a direction, a wavelength, an intensity, etc. of the light emitted from each microlens 121.

In this manner, in the light-ray reproduction type display device 15, each microlens 121 constitutes a light emission point, and the light emitted from each light emission point is controlled by a plurality of pixels 111 provided immediately below each microlens 121. By driving each pixel 111 on the basis of the light-ray information, the light emitted from each light emission point is controlled and a desired light-ray state is implemented.

Specifically, in the example illustrated in, for example, FIG. 4, the light-ray information includes information about an emission state of light (a direction, a wavelength, an intensity, etc. of emitted light) in each microlens 121 for observing an image (that is, a virtual image) on a virtual display surface located at the position X different from the position O when the real display surface located at the position O (corresponding to the display surface 125 of the microlens array 120 illustrated in FIG. 5) is viewed. Each pixel 111 is driven on the basis of the light-ray information and light whose emission state is controlled is emitted from each microlens 121, so that the user's pupil is irradiated with light for observing a virtual image at the position X for the user located at the observation position. It can also be said that controlling the emission state of light on the basis of the light-ray information is controlling the irradiation state of light for the user's pupil.

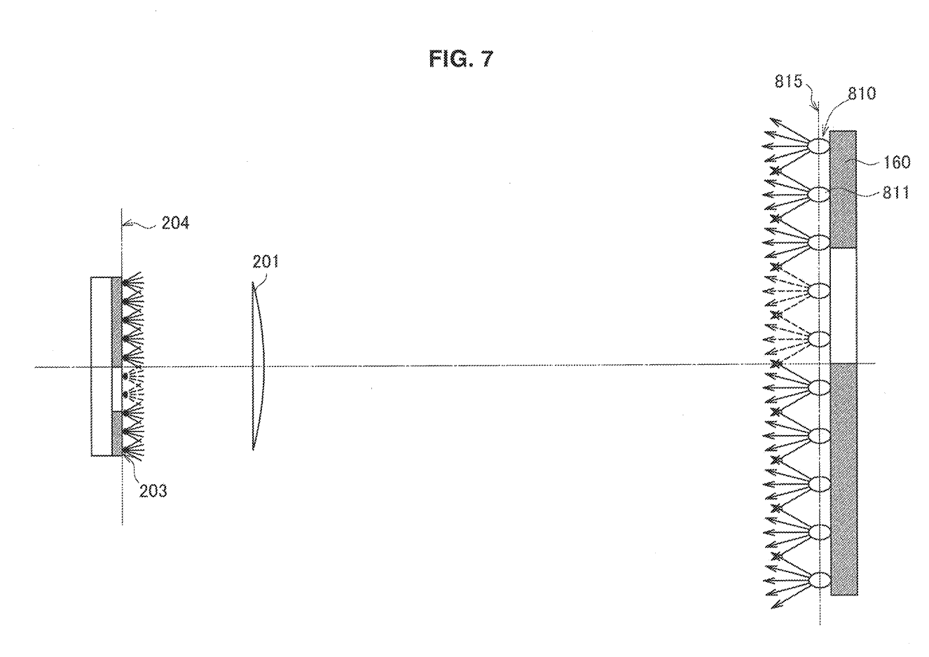

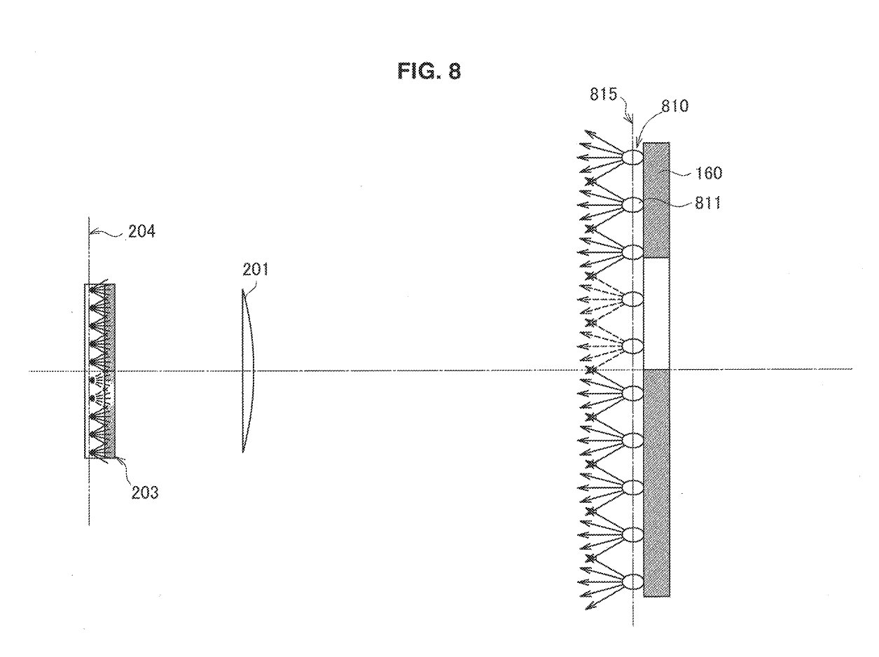

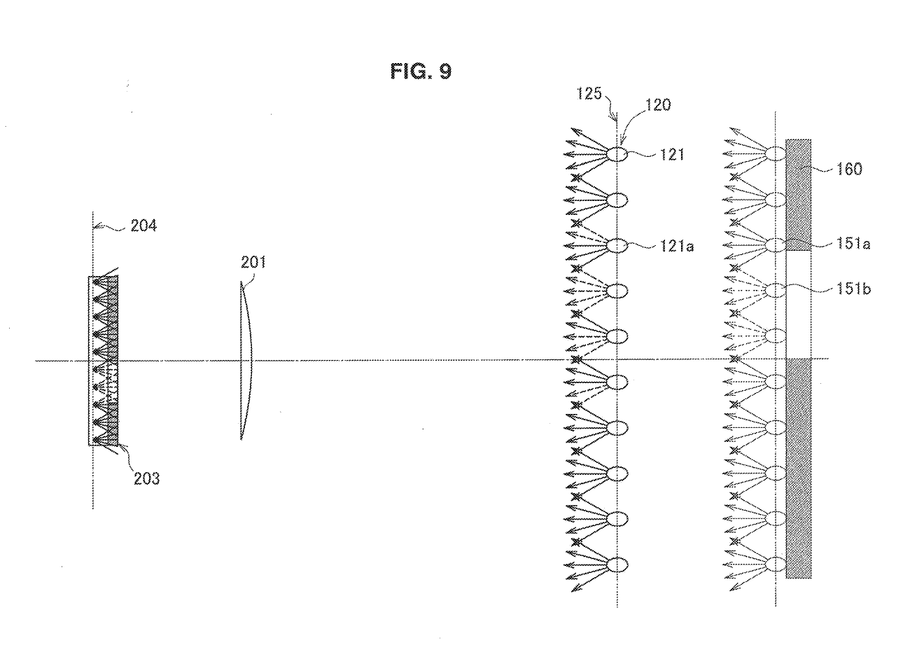

The above-described details including the state of image formation on the retina of the user will be described in more detail with reference to FIGS. 7 to 9. FIG. 7 is a schematic diagram illustrating a state in which the user's focus is aligned with the display surface in the general two-dimensional display device 80. FIG. 8 is a schematic diagram illustrating a state in which the user's focus is not aligned with the display surface in the general two-dimensional display device 80. FIG. 9 is a schematic diagram illustrating a relationship between a virtual image surface in the light-ray reproduction type display device 15 and an image formation surface on the user's retina. In FIGS. 7 to 9, the pixel array 810 and the display surface 815 of the general two-dimensional display device 80 or the microlens array 120 and the display surface 125 of the light-ray reproduction type display device 15 and a lens 201 (a crystalline lens 201) and a retina 203 of an eye of the user are schematically illustrated.

Referring to FIG. 7, a state in which a picture 160 is displayed on the display surface 815 is schematically illustrated. In the general two-dimensional display device 80, in a state in which the user's focus is aligned with the display surface 815, light from each pixel 811 of the pixel array 810 passes through the lens 201 of the user's eye and an image thereof is formed on the retina 203 (that is, the image formation surface 204 is located on the retina 203). Arrows drawn with different line types in FIG. 7 indicate light of different wavelengths emitted from the pixels 811, that is, light of different colors.

In FIG. 8, a state in which the display surface 815 is located closer to the user than in the state illustrated in FIG. 7 and the user's focus is not aligned with the display surface 815 is illustrated. Referring to FIG. 8, light from each pixel 811 of the pixel array 810 does not form an image on the user's retina 203 and the image formation surface 204 is located behind the retina 203. In this case, a blurred picture out of focus is recognized by the user. FIG. 8 illustrates a state in which a user having presbyopia views a blurred picture in an attempt to view a nearby display surface.

FIG. 9 illustrates a light-ray state when the light-ray reproduction type display device 15 is driven such that it displays a picture 160 on the virtual image surface 150 as a virtual image for the user. In FIG. 9, similar to the display surface 815 illustrated in FIG. 8, the display surface 125 is located relatively close to the user. The virtual image surface 150 is set as a virtual display surface located farther away than the real display surface 125.

Here, as described above, in the light-ray reproduction type display device 15, an emission state of light can be controlled so that microlenses 121 (that is, light emission points 121) emit light of mutually different light intensities and/or wavelengths in mutually different directions instead of isotropically emitting unique light. For example, the light emitted from each microlens 121 is controlled so that the light from the picture 160 on the virtual image surface 150 is reproduced. Specifically, for example, assuming virtual pixels 151 (151a and 151b) on the virtual image surface 150, it can be considered that light of a first wavelength is emitted from a certain virtual pixel 151a and light of a second wavelength is emitted from the other virtual pixel 151b in order to display the picture 160 on the virtual image surface 150. In accordance with this, the emission state of the light is controlled so that the microlens 121a emits the light of the first wavelength in the direction corresponding to the light from the pixel 151a and emits the light of the second wavelength in the direction corresponding to the light from the pixel 151b. Although not illustrated, a pixel array is actually provided on the back side (the right side of the drawing sheet in FIG. 9) of the microlens array 120 as illustrated in FIG. 5 and driving of each pixel of the pixel array is controlled, so that the emission state of light from the microlens 121a is controlled.

Here, the distance from the retina 203 of the virtual image surface 150 is set to a position in focus for the user, for example, a position of the display surface 815 illustrated in FIG. 7. The light-ray reproduction type display device 15 is driven such that it reproduces the light from the picture 160 on the virtual image surface 150 located at such a position, so that the image formation surface 204 of the light from the real display surface 125 is located behind the retina 203, but an image of the picture 160 on the virtual image surface 150 is formed on the retina 203. Accordingly, in terms of a user having presbyopia, even when the distance between the user and the display surface 125 is short, the user can view a favorable picture 160 similar to that in a distant view.

The basic principle of the present embodiment has been described above. As described above, in the present embodiment, by using the light-ray reproduction type display device, the light from the picture 160 on the virtual image surface 150 which is set at a position in focus for a user with presbyopia is reproduced and the light is emitted to the user. This allows the user to observe the in-focus picture 160 on the virtual image surface 150. Accordingly, for example, even when the picture 160 is a high-resolution picture in which the resolution at the viewing distance on the real display surface 125 exceeds the limit resolution of the user, the in-focus picture is provided to the user without using additional optical compensation instruments such as presbyopic glasses and a fine picture 160 can be observed. Consequently, even when the density of information is increased in a comparatively small display screen as described in the above (1. Background of present disclosure), the user can favorably observe a picture on which high-density information is displayed by supplementing the visual acuity of the user. Also, according to the present embodiment, because it is possible to perform display in which visual acuity compensation is performed without using optical compensation instruments such as presbyopic glasses as described above, it is unnecessary to carry additional portable items such as presbyopic glasses themselves and/or a glasses case for storing presbyopic glasses and the burden on the user is decreased.

Also, although a case in which the virtual image surface 150 is set to be farther away than the real display surface 125 as illustrated in FIG. 9 to compensate for the visual acuity for the user with presbyopia has been described above, the present embodiment is not limited to such an example. For example, the virtual image surface 150 may be set to be closer than the real display surface 125. In this case, the virtual image surface 150 is set at a position in focus for, for example, a user with myopia. Thereby, the user with myopia can observe the in-focus picture 160 without using optical compensation instruments such as eyeglasses and contact lenses. Switching of display between visual acuity compensation for a user with presbyopia and visual acuity compensation for a user with myopia can be freely implemented simply by changing data displayed on each pixel and it is unnecessary to change a hardware mechanism.

### 3. Display Device According to Present Embodiment

A detailed configuration of the display device according to the present embodiment capable of implementing an operation based on the basic principle described above will be described.

### (3-1. Device Configuration)

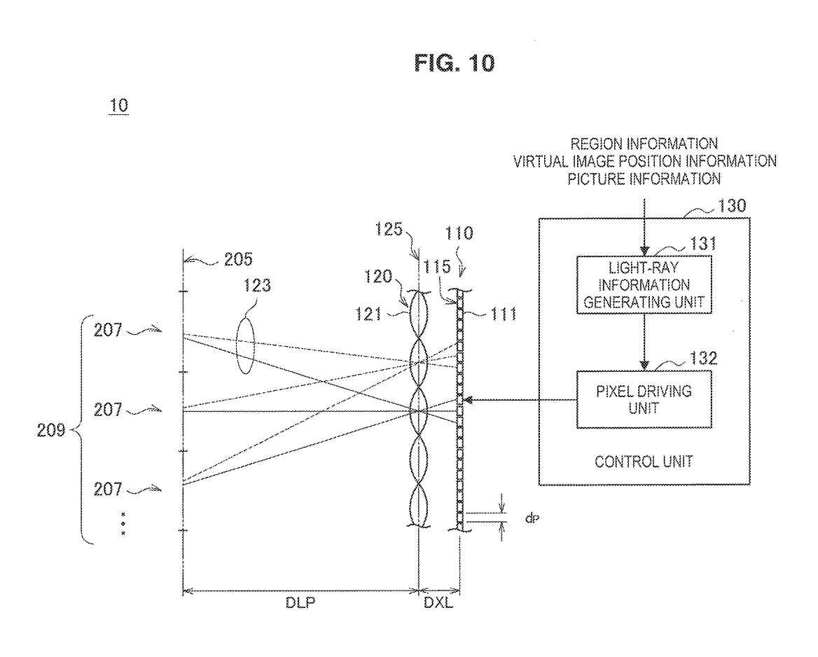

The configuration of the display device according to the present embodiment will be described with reference to FIG. 10. FIG. 10 is a diagram illustrating an example of the configuration of the display device according to the present embodiment.

Referring to FIG. 10, the display device 10 according to the present embodiment includes a pixel array 110 in which a plurality of pixels 111 are two-dimensionally arranged, a microlens array 120 provided on the display surface 115 of the pixel array 110, and a control unit 130 that controls driving of each pixel 111 of the pixel array 110. Here, the pixel array 110 and the microlens array 120 illustrated in FIG. 10 are similar to those illustrated in FIG. 5. Also, the control unit 130 drives each pixel 111 such that it reproduces a predetermined light-ray state on the basis of the light-ray information. In this manner, the display device 10 can be configured as a light-ray reproduction display device.

As in the light-ray reproduction type display device 15 described with reference to FIG. 5, the pitch of the microlenses 121 in the microlens array 120 is configured to be larger than the pitch of the pixels 111 in the pixel array 110 and light from a plurality of pixels 111 is incident on one microlens 121 and is emitted with directivity. As described above, in the display device 10, each microlens 121 constitutes a light emission point. The microlens 121 corresponds to a pixel in a general two-dimensional display device, and the lens surface 125 of the microlens array 120 becomes an apparent display surface 125 in the display device 10.

The pixel array 110 may include a liquid crystal layer (liquid crystal panel) of a liquid crystal display device having, for example, a pixel pitch of about 10 (μm). Although not illustrated, various structures provided for the pixels in general liquid crystal display devices such as a driving element for driving each pixel of the pixel array 110 and a light source (backlight) may be connected to the pixel array 110. However, the present embodiment is not limited to this example and another display device such as an organic EL display device or the like may be used as the pixel array 110. Also, the pixel pitch is not limited to the above example and may be appropriately designed in consideration of the resolution etc. desired to be implemented.

The microlens array 120 is configured by two-dimensionally arranging convex lenses having, for example, a focal length of 3.5 (mm), in a lattice form with a pitch of 0.15 (mm). The microlens array 120 is provided to substantially cover the entire pixel array 110. The pixel array 110 and the microlens array 120 are configured to be at positions at which an image on the display surface 115 of the pixel array 110 is approximately formed on a plane substantially parallel to the display surface 115 (or the display surface 125) including the user's pupil. Generally, the image formation position of the picture on the display surface 115 can be preset as an observation position assumed when the user observes the display surface 115. However, the focal length and the pitch of the microlenses 121 in the microlens array 120 are not limited to the above-described example, and may be appropriately designed on the basis of an arrangement relationship with other members, the image formation position of the picture on the display surface 115 (that is, an assumed observation position of the user), or the like.

The control unit 130 includes a processor such as a central processing unit (CPU) or a digital signal processor (DSP) and operates in accordance with a predetermined program, thereby controlling the driving of each pixel 111 of the pixel array 110. The control unit 130 has a light-ray information generating unit 131 and a pixel driving unit 132 as its functions.

The light-ray information generating unit 131 generates light-ray information on the basis of region information, virtual image position information, and picture information. Here, the region information is information about a region group including a plurality of regions which are set on a plane including the user's pupil and substantially parallel to the display surface 125 of the microlens array 120 and which are smaller than the pupil diameter of the user. The region information includes information about a distance between the plane on which the region is set and the display surface 125, information about a size of the region, and the like.

In FIG. 10, a plane 205 including the pupil of the user, a plurality of regions 207 set on the plane 205, and a region group 209 are simply illustrated. The plurality of regions 207 are set to be located in the pupil of the user. The region group 209 is set in a range in which light emitted from each microlens 121 can reach the plane 205. In other words, the microlens array 120 is configured so that the region group 209 is irradiated with the light emitted from one microlens 121.

Here, in the present embodiment, the wavelength, the intensity, and the like of light emitted from each microlens 121 are adjusted in accordance with the combination of the microlens 121 and the region 207. That is, for each region 207, the irradiation state of light incident on the region 207 is controlled. The region 207 corresponds to a size in which light from one pixel 111 is projected onto the pupil (a projection size of light from the pixel 111 on the pupil) and an interval between the regions 207 can be said to indicate a sampling interval when light is incident on the pupil of the user. In the following description, the region 207 is also referred to as a sampling region 207. The region group 209 is also referred to as a sampling region group 209.

The virtual image position information is information about a position at which a virtual image is generated (a virtual image generation position). The virtual image generation position is the position of the virtual image surface 150 illustrated in FIG. 9. The virtual image position information includes information about the distance from the display surface 125 to the virtual image generation position. Also, the picture information is two-dimensional picture information presented to the user.

On the basis of the region information, the virtual image position information, and the picture information, the light-ray information generating unit 131 generates light-ray information indicating the light-ray state for light from the picture to be incident on each sampling region 207 based on the region information when the picture based on the picture information is displayed at the virtual image generation position based on the virtual image position information. The light-ray information includes information about the emission state of light in each microlens 121 and information about the irradiation state of the light for each sampling region 207 for reproducing the light-ray state. A process to be performed by the light-ray information generating unit 131 corresponds to a process of assigning depth information to the two-dimensional picture information described with reference to FIG. 4 in the above (2. Basic principle of present embodiment).

Also, the picture information may be transmitted from another device or may be pre-stored in a storage device (not shown) provided in the display device 10. The picture information may be information about pictures, text, graphs, and the like which represent results of various processes executed by a general information processing device.

Also, the virtual image position information may be input in advance by, for example, the user, a designer of the display device 10, or the like, and stored in the above-described storage device. Also, in the virtual image position information, the virtual image generation position is set to be a position in focus for the user. For example, a general focus position that is suitable for a relatively large number of users having presbyopia may be set as a virtual image generation position by the designer of the display device 10 or the like. Alternatively, the virtual image generation position may be appropriately adjusted in accordance with the user's visual acuity by the user, and the virtual image position information within the above-described storage device may be updated each time.

Also, the region information may be input in advance by, for example, the user, the designer of the display device 10, or the like, and may be stored in the above-described storage device. Here, the distance between the display surface 125 and a plane 205 on which the sampling region 207 is set (the plane 205 corresponds to the observation position of the user) included in the region information may be set on the basis of a position at which the user is assumed to generally observe the display device 10. For example, if a device equipped with the display device 10 is a wristwatch type wearable device, the above-described distance can be set in consideration of a distance between the user's pupil and an arm that is an attachment position of the wearable device. Also, for example, if the device equipped with the display device 10 is a stationary type television installed in a room, the above-described distance can be set in consideration of a general distance between a television and a user's pupil when the television is watched. Alternatively, the above-described distance may be appropriately adjusted by the user in accordance with a usage mode, and the virtual image position information in the storage device may be updated each time. Also, the size of the sampling region 207 included in the region information can be appropriately set in consideration of matters to be described in the following (3-3-1. Sampling region).

The light-ray information generating unit 131 provides the generated light-ray information to the pixel driving unit 132.

The pixel driving unit 132 drives each pixel 111 of the pixel array 110 such that it reproduces the light-ray state when a picture based on the picture information is displayed on the virtual image surface on the basis of the light-ray information. At this time, the pixel driving unit 132 drives each pixel 111 so that the light emitted from each microlens 121 is controlled independently for each sampling region 207. Thereby, as described above, the irradiation state of light incident on the sampling region 207 is controlled for each sampling region 207. For example, in the example illustrated in FIG. 10, a state in which light 123 configured by superimposing light from a plurality of pixels 111 is incident on each sampling region 207 is illustrated.

Here, the projection size of the light 123 on the pupil (on the plane 205) needs to be equal to or less than the size of the sampling region 207 in order to cause the light 123 to be incident on the sampling region 207. Accordingly, in the display device 10, the structure, arrangement, and the like of each member are designed so that the projection size of the light 123 on the pupil is equal to or smaller than the size of the sampling region 207.

On the other hand, as will be described in detail in the following (2-2-3-1. Sampling region), an amount of blur of the image on the retina of the user depends upon the projection size of the light 123 on the pupil (that is, an entrance pupil diameter of light). If the amount of blur on the retina is larger than the size on the retina of an image capable of being distinguished by the user, a blurred image will be recognized by the user. When an adjustment function of the eye is insufficient due to presbyopia or the like, the projection size of the light 123 on the pupil corresponding to the size of the sampling region 207 needs to be sufficiently smaller than the pupil diameter in order to make the amount of blur on the retina equal to or smaller than the size on the retina of an image capable of being distinguished by the user.

Specifically, whereas the general human pupil diameter is about 2 (mm) to 8 (mm), it is preferable to set the size of the sampling region 207 to about 0.6 (mm) or less. Conditions required for the size of the sampling region 207 will be described in detail again in the following

### (3-3-1. Sampling Region).

Here, as is apparent from FIG. 10, the projection size of the light 123 on the pupil depends on an image magnification and a size dp of the pixel 111 of the pixel array 110. Here, the image magnification is a ratio between a viewing distance (a distance between the lens surface 125 of the microlens array 120 and the pupil) DLP and a distance DXL between the lens surface 125 of the microlens array 120 and the display surface 115 of the pixel array 110 (DLP/DXL). Accordingly, in the present embodiment, the size dp of the pixel 111, the arrangement positions of the microlens array 120 and the pixel array 110, and the like may be appropriately designed so that the projection size of the light 123 on the pupil is sufficiently smaller than the pupil diameter (in more detail, about 0.6 (mm) or less) in consideration of a distance (that is, DLP) at which the user is assumed to generally observe the display surface 125.

Also, in the display device 10, the arrangement of each constituent member is set so that the irradiation state of light with respect to each sampling region 207 is periodically iterated in units larger than the maximum pupil diameter of the user. This is for displaying a picture similar to that before a movement to a user even at a position after a movement of the user's pupil position when the position of the pupil of the user has moved. The iteration cycle is determined by the pitch of the microlenses 121 of the microlens array 120, DXL, and DLP. Specifically, iteration cycle=(pitch of microlens array 120)×(DLP+DXL)/DXL. On the basis of this relationship, the pitch of the microlenses 121, the size dp and the pitch of the pixels 111 in the pixel array 110, and values such as DXL and DLP are set so that the iteration cycle satisfies the above-described conditions. The conditions required for the iteration cycle will be described in detail again in the following (3-3-2. Iteration cycle of irradiation state of sampling region).

As described above, the configuration of the display device 10 according to the present embodiment has been described with reference to FIG. 10.

Here, the display device 10 according to the present embodiment is similar to a light-ray reproduction type display device widely used as a naked-eye 3D display device in terms of a partial configuration. However, because an objective of the naked-eye 3D display device is to display a picture having binocular parallax with respect to the left and right eyes of the user, the emission state of emitted light is controlled only in the horizontal direction and the control of the emission state is not performed in the vertical direction in many cases. Accordingly, for example, in many cases, a configuration in which a lenticular lens is provided on the display surface of the pixel array is provided. On the other hand, because an objective of the display device 10 according to the present embodiment is to display a virtual image for the purpose of compensating for the eye adjustment function for the user, the control of the emission state is naturally performed in both directions of the horizontal direction and the vertical direction. Thus, instead of the lenticular lens as described above, the microlens array 120 in which the microlenses 121 are two-dimensionally arranged is used on the display surface of the pixel array.

Also, because an objective of the naked-eye 3D display device is to display a picture having binocular parallax with respect to the left and right eyes of the user as described above, the sampling region 207 described in the present embodiment is set as a relatively large region including the whole eye of the user. Specifically, the size of the sampling region 207 is set to about 65 (mm), which is the average value of a user's pupil distance (PD), or about a fraction thereof in many cases. On the other hand, in the present embodiment, the size of the sampling region 207 is set to be smaller than the pupil diameter of the user, in more detail, smaller than about 0.6 (mm). As described above, because the purpose and the field of application are different, a structure different from that of a general naked eye 3D display device is adopted and different drive control is performed in the display device 10 according to the present embodiment.

### (3-2. Driving Example)

Next, a specific driving example in the display device 10 illustrated in FIG. 10 will be described. The display device 10 according to the present embodiment can be driven in a mode in which a virtual image on a virtual display surface different from the real display surface 125 is displayed (that is, picture information to which depth information is assigned is displayed) (hereinafter also referred to as a visual acuity compensation mode) or a mode in which two-dimensional picture information is displayed (hereinafter also referred to as a normal mode). Because the virtual image is visually recognized by a user in the visual acuity compensation mode, it is possible to provide a favorable picture even for a user for which it is difficult to align a focus on the real display surface 125 due to presbyopia or myopia. On the other hand, in the normal mode, it is possible to display, for example, a two-dimensional picture similar to that of the general two-dimensional display device 80 illustrated in FIG. 6, with the configuration of the display device 10 illustrated in FIG. 10.

### (3-2-1. Normal Mode)

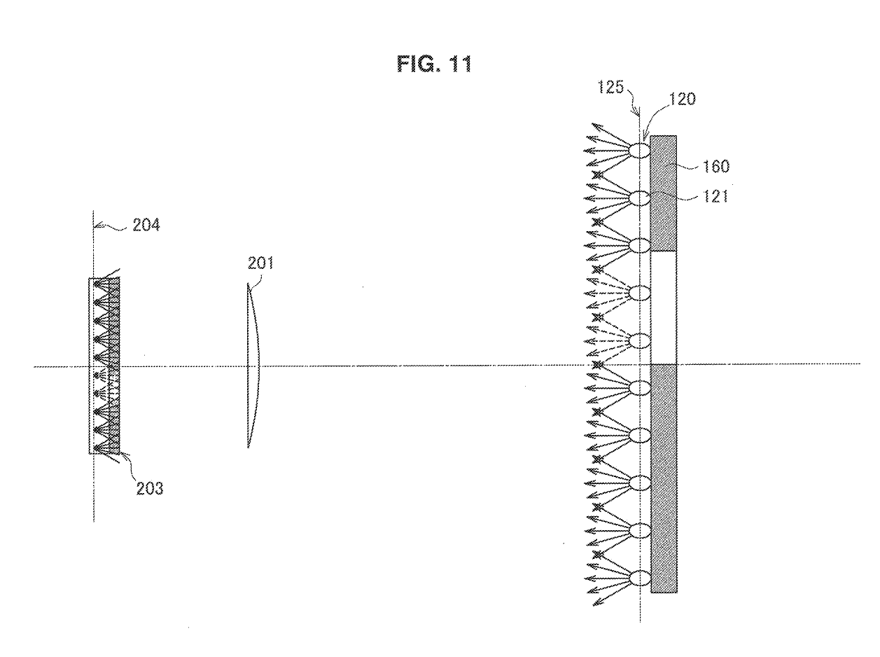

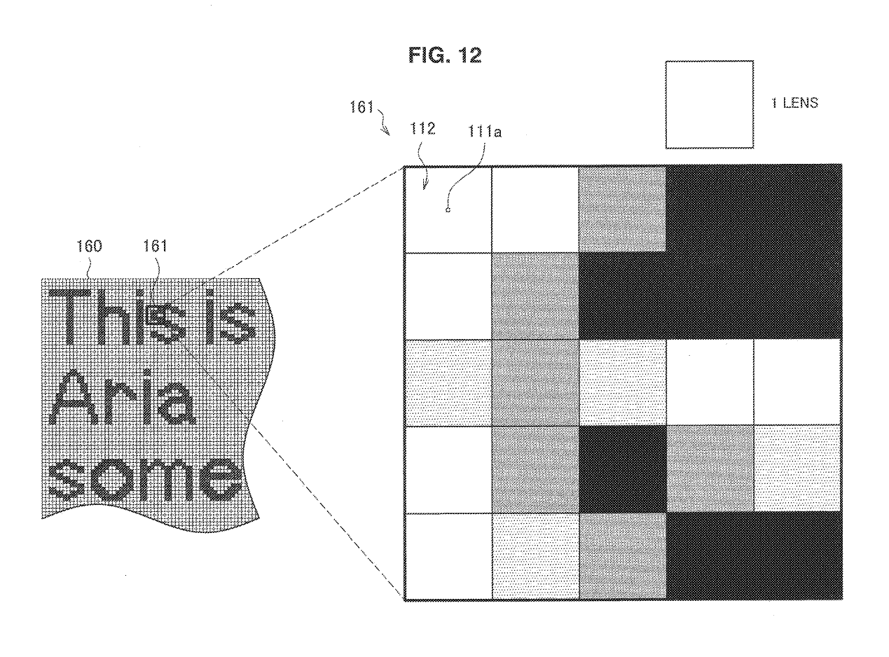

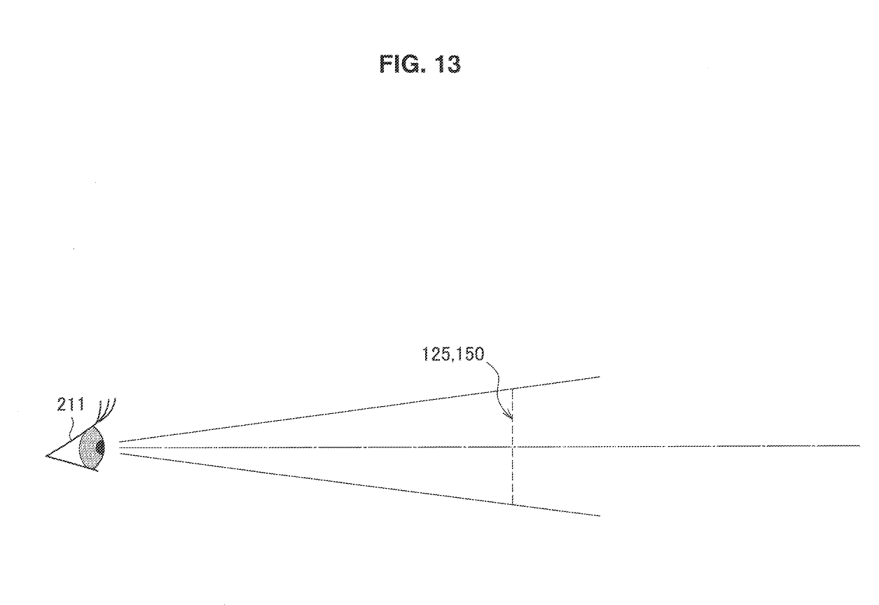

Driving of the display device 10 in the normal mode will be described with reference to FIGS. 11 to 13. FIG. 11 is a diagram illustrating light rays emitted from the microlens 121 in the normal mode. FIG. 12 is a diagram illustrating a specific display example of the pixel array 110 in the normal mode. FIG. 13 is a diagram illustrating a positional relationship between the virtual image surface 150 and the display surface 125 of the microlens array 120 in the normal mode.

Referring to FIG. 11, as in FIG. 9, the microlens array 120 and the display surface 125 thereof, the user's eye lens 201, and the user's retina 203 are schematically illustrated. Also, the picture 160 displayed on the display surface 125 is schematically illustrated. Also, FIG. 11 corresponds to an example in which the picture 160 reproduced by the pixel array 810 in FIG. 8 described above is reproduced by a configuration similar to that in the present embodiment illustrated in FIG. 9. Accordingly, repeated description of matters already described with reference to FIG. 8 and FIG. 9 will be omitted.

As illustrated in FIG. 11, in the normal mode, the same light is emitted from each microlens 121 in directions of all emission angles. Thereby, each microlens 121 behaves as in each pixel 811 of the pixel array 810 illustrated in FIG. 8 and the picture 160 is displayed on the display surface 125 of the microlens array 120 by the microlens array 120.

FIG. 12 illustrates an example of a picture 160 that the user can actually visually recognize in the normal mode and a state in which a partial region of the pixel array 110 when the picture 160 is being displayed is enlarged. For example, as illustrated in FIG. 12, in the normal mode, the user is assumed to visually recognize the picture 160 including predetermined text data.

Here, the picture 160 in FIG. 12 is actually recognized by the user when the user sees the light from the pixel array 110 via the microlens array 120. An illustration obtained by enlarging a partial region 161 of the picture 160 and removing the microlens array 120 (that is, an illustration of the display of the pixel array 110 immediately below the region 161) is illustrated on the right side in FIG. 12. A pixel group 112 including a plurality of pixels 111 is located immediately below one microlens 121, but the same information is displayed in a pixel group 112 located immediately below one microlens 121 in the normal mode as illustrated on the right side of FIG. 12.

In this manner, each pixel 111 is driven so that the same information is displayed in the pixel group 112 immediately below each microlens 121 in the normal mode, so that two-dimensional picture information is displayed on the display surface 125 of the microlens array 120. The user can visually recognize a two-dimensional picture existing on the display surface 125 similar to the picture 160 provided in the general two-dimensional display device as illustrated in FIG. 8.

FIG. 13 illustrates relationships between the user's eye 211, the display surface 125 of the microlens array 120, and the virtual image surface 150. The normal mode corresponds to a state in which the virtual image surface 150 and the display surface 125 of the microlens array 120 coincide as illustrated in FIG. 13.

### (3-2-2. Visual Acuity Compensation Mode)

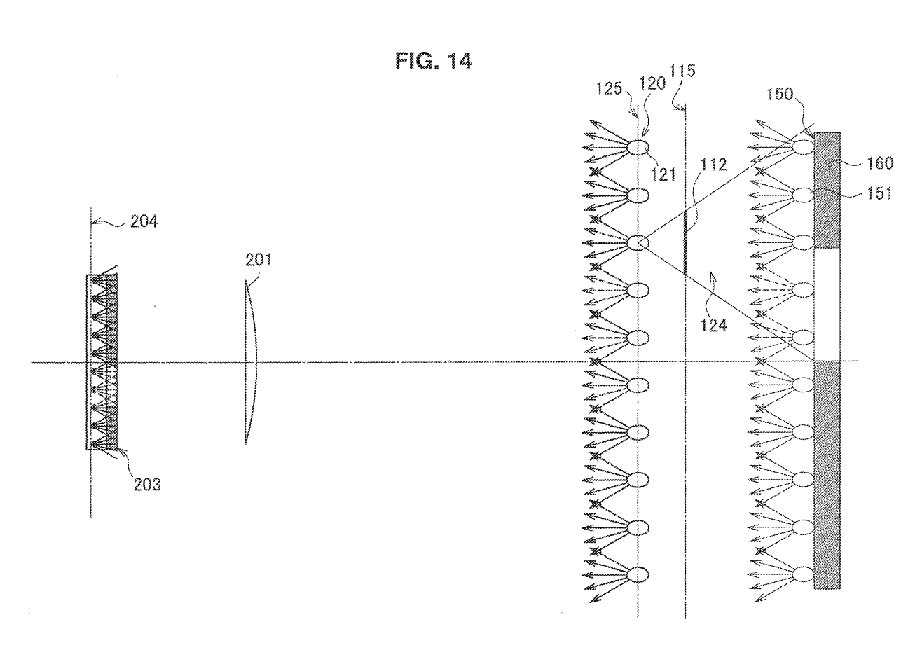

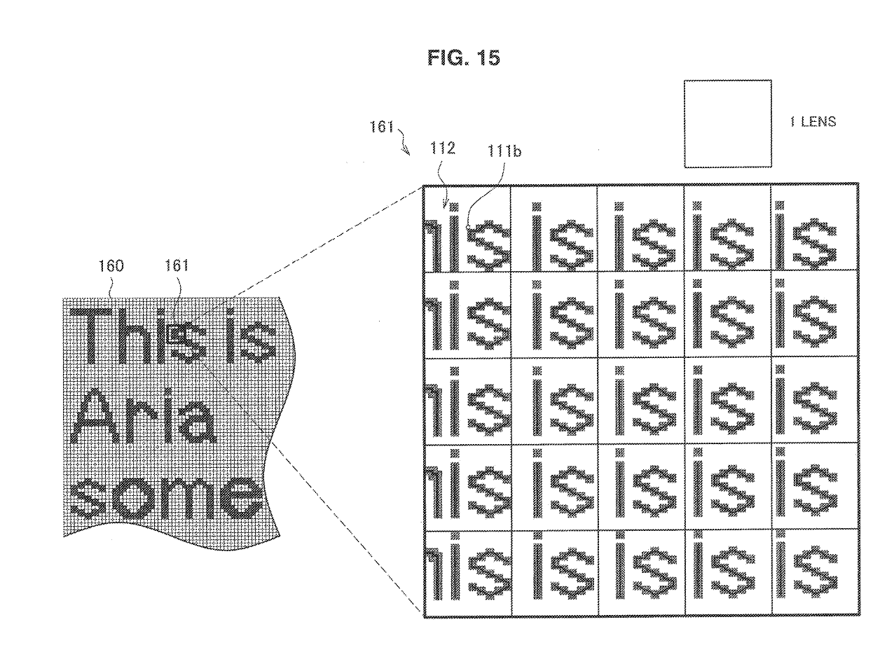

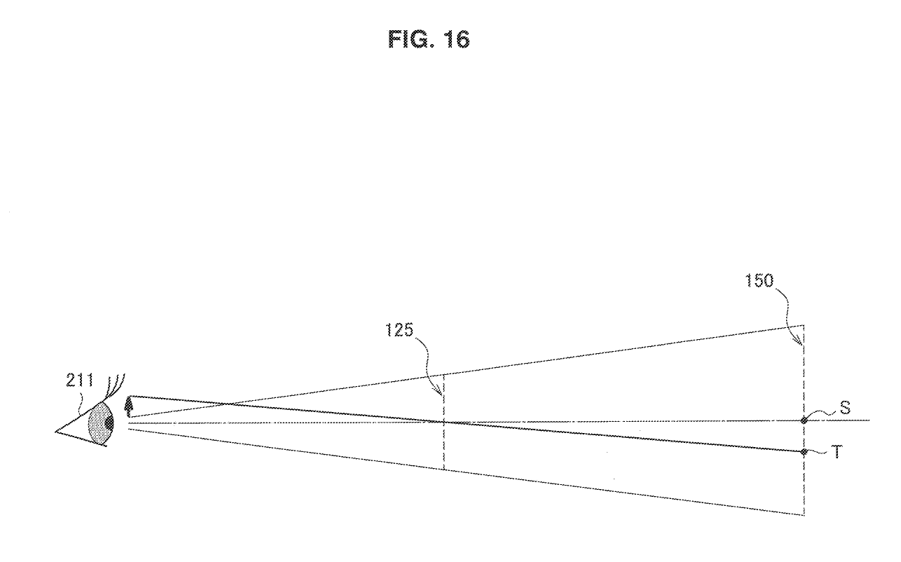

Next, the driving of the display device 10 in the visual acuity compensation mode will be described with reference to FIGS. 14 to 16. FIG. 14 is a diagram illustrating light rays emitted from the microlens 121 in the visual acuity compensation mode. FIG. 15 is a diagram illustrating a specific display example of the pixel array 110 in the visual acuity compensation mode. FIG. 16 is a diagram illustrating a positional relationship between the virtual image surface 150 and the display surface 125 of the microlens array 120 in the visual acuity compensation mode.

Referring to FIG. 14, as in FIG. 9, the microlens array 120 and the display surface 125 thereof, the virtual image surface 150, the virtual pixels 151 on the virtual image surface 150, the picture 160 on the virtual image surface, the lens 201 of the eye of the user, and the user's retina 203 are schematically illustrated. Also, in FIG. 14, the display surface 115 of the pixel array 110, which is not illustrated in FIG. 9, is also illustrated.

Also, FIG. 14 corresponds to an illustration obtained by adding the display surface 115 of the pixel array 110 to FIG. 9 described above. Accordingly, repeated description of matters already described with reference to FIG. 9 will be omitted.

In the visual acuity compensation mode, light is emitted from each microlens 121 to reproduce the light from the picture 160 on the virtual image surface 150. The picture 160 can be considered as a two-dimensional picture on the virtual image surface 150 displayed by the virtual pixels 151 on the virtual image surface 150. A range 124 of light that can be independently controlled in one certain microlens 121 is schematically illustrated in FIG. 14. The pixel group 112 (a part of the pixel array 110) immediately below the microlens 121 is driven such that the light from the virtual pixels 151 is reproduced on the virtual image surface 150 included in the range 124. Similar drive control is performed in each microlens 121, so that light is emitted from each microlens 121 to reproduce the light from the picture 160 on the virtual image surface 150.

An example of a picture 160 capable of being actually visually recognized by the user in the visual acuity compensation mode and a state in which a partial region of the pixel array 110 when the picture 160 is being displayed is enlarged are illustrated in FIG. 15. For example, as illustrated in FIG. 15, the user is assumed to visually recognize the picture 160 including predetermined text data. In the visual acuity compensation mode, the picture 160 is visually recognized by the user as a picture displayed on the virtual image surface 150 illustrated in FIG. 14.

Here, the picture 160 in FIG. 15 is actually recognized by the user when the user views the light from the pixel array 110 via the microlens array 120. An illustration obtained by enlarging a partial region 161 of the picture 160 and removing the microlens array 120 (that is, an illustration of the display of the pixel array 110 immediately below the region 161) is illustrated on the right side in FIG. 15.

A pixel group 112 including a plurality of pixels 111 is located immediately below one microlens 121. As illustrated in the drawing on the right side of FIG. 15, in the pixel group 112 located immediately below each microlens 121, the same information as in the normal mode is displayed in pixels located on an extension of the center of the microlens 121 when viewed from a certain point (that is, the same information is displayed on the pixel 111a illustrated in FIG. 12 and the pixel 111b illustrated in FIG. 15), but picture information that can be viewed through the movement of the viewpoint of the user is displayed around the pixels 111a and 111b.

Relationships between the user's eye 211, the display surface 125 of the microlens array 120, and the virtual image surface 150 are illustrated in FIG. 16. As illustrated in FIG. 16, in the visual acuity compensation mode, the virtual image surface 150 is located farther away than the display surface 125 by the microlens array 120. In FIG. 16, the movement of the viewpoint of the user is indicated by an arrow. In consideration of movement of the point visually recognized by the user on the virtual image surface 150 (movement from a point S to a point T in FIG. 16) corresponding to the movement of the user's viewpoint, picture information that can be viewed through the movement of the viewpoint is displayed on the pixel group 112 immediately below the microlens 121 as illustrated in FIG. 15. Each pixel 111 is driven as described above, so that the picture 160 is displayed to the user as if it were located on the virtual image surface 150.

Examples of driving in the normal mode and the visual acuity compensation mode have been described above as an example of driving in the display device 10.

### (3-3. Detailed Design)

A more detailed design method for each configuration in the display device 10 illustrated in FIG. 10 will be described. Here, conditions required for the size of the sampling region 207 illustrated in FIG. 10 and conditions required for the iteration cycle of the irradiation state of light for each sampling region 207 will be described.

### (3-3-1. Sampling Region)

As described above, it is preferable that the size of the sampling region 207 be sufficiently small with respect to the pupil diameter of the user so that a favorable image without blur is provided to the user. Hereinafter, the conditions required for the size of the sampling region 207 will be specifically examined.

For example, a level at which presbyopia can be first recognized is about 1 D (Diopter) as the strength of a necessary correction lens (presbyopic glasses). Here, if a Listing model obtained by modeling an average eyeball is used, the eyeball can be regarded to include a single lens of 60 D and a retina located at a distance of 22.22 (mm) from the single lens.

Light is incident on the retina via a lens of 60 D−1 D=59 D for the user wearing presbyopic glasses with an intensity of 1 D described above, so that the image formation surface can be formed at a position of 22.22×(60 D/59 D−1)≈0.38 (mm) behind the retina in the eyeball of the user. Also, in this case, when the entrance pupil diameter of light (corresponding to the projection size of the light 123 on the pupil illustrated in FIG. 10) is Ip, an amount of blur on the retina being Ip×0.38/22.22 (mm) can be obtained.

Here, when the visual acuity required for practical use is 0.5, the size of the image on the retina to be distinguished is about 0.0097 (mm) from the calculation shown in the following Equation (1). In the following Equation (1), 1.33 is a refractive index in the eyeball.

[Math. 1]

(1/(0.5×60))×(π/180)×22.22/1.33≅0.0097 (mm) (1)

If the amount of blur on the retina is smaller than the size of the image on the retina to be distinguished, the user can observe a clear image without blur. If Ip is obtained so that the above-described amount of blur on the retina (Ip×0.38/22.22 (mm)) is the size (0.0097 (mm)) of the image on the retina to be distinguished, Ip is about 0.6 (mm) from the following Equation (2).

[Math. 2]

Ip=0.0097×22.22/0.38≅0.6 (mm) (2)

When the degree of presbyopia is stronger, the distance of 0.38 (mm) between the retina and the image formation surface described above becomes longer, so that Ip becomes smaller from the above-described Equation (2). Also, when the required visual acuity is larger, a larger value is substituted for “0.5” in the above-described Equation (1), so that the size of the image on the retina to be distinguished is smaller than the above-described value (0.0097 (mm)) and Ip becomes smaller from the above-described Equation (2). Accordingly, it can be said that Ip 0.6 (mm) calculated from the above-described Equation (2) substantially corresponds to a lower limit value required for an entrance pupil diameter of light.

In the present embodiment, because the light incident on each sampling region 207 is controlled, the size of the sampling region 207 is determined depending on the entrance pupil diameter of light. Accordingly, it can also be said that Ip 0.6 (mm) calculated from the above-described Equation (2) is the lower limit value of the sampling region 207. As described above, in the present embodiment, the sampling region 207 is preferably set so that its size is 0.6 (mm) or less.

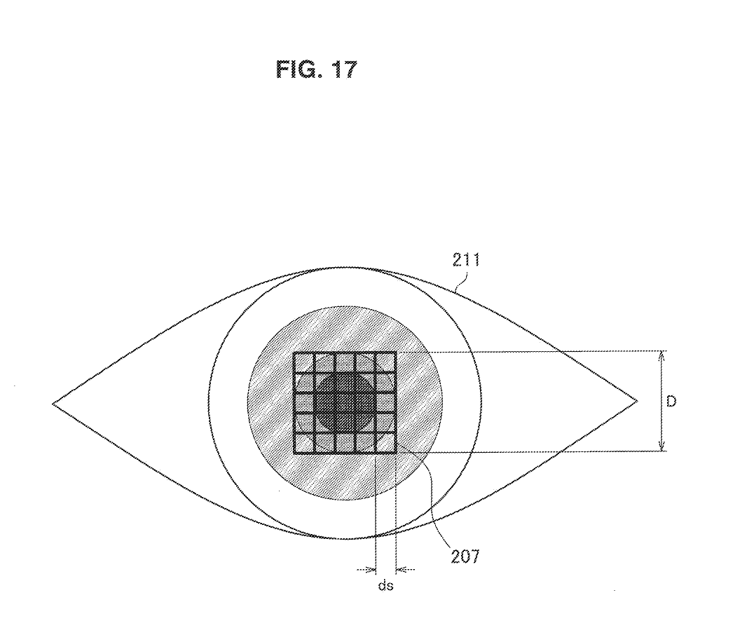

FIG. 17 is a diagram illustrating a relationship between the pupil diameter of the user's pupil and the size of the sampling region 207. In FIG. 17, the sampling region 207 set on the pupil of the user together with the user's eye 211 is schematically illustrated. A general human pupil diameter D is known to be about 2 (mm) to 8 (mm). On the other hand, as described above, a size ds of the sampling region 207 is preferably 0.6 (mm) or less. Accordingly, in the present embodiment, as illustrated in FIG. 17, a plurality of regions 207 are set in the pupil. Although a case in which the shape of the sampling region 207 is square has been described here, the shape of the sampling region 207 may be any of other various shapes such as a hexagon and a rectangle if the above-described conditions of the size are satisfied.

The conditions required for the size of the sampling region 207 have been described above.

Here, in the above-described Patent Literature 1, a configuration in which light from a plurality of pixels is emitted from each of a plurality of microlenses and projected onto the pupil of the user is also disclosed. However, in the technology described in Patent Literature 1, only one of projected images of light corresponding to pixels is incident on the user's pupil. This corresponds to the state in which only one sampling region 207 smaller than the pupil diameter is provided on the pupil at an interval equal to or larger than the pupil diameter in the present embodiment.

In the technology described in the above-described Patent Literature 1, blur is decreased by decreasing a size of a light beam incident on the pupil without performing a process of obtaining the light beam being incident on different points on the pupil through the virtual image generation process as in the present embodiment. Accordingly, when a plurality of light beams are incident on the pupil from the same lens, blur occurs in the image on the retina. Accordingly, in the technology described in the above-described Patent Literature 1, the interval of the light incident on the plane 205 including the pupil, that is, the interval at which the sampling regions 207 are provided is adjusted to be larger than the pupil diameter.

However, in this configuration, there is inevitably a moment when light is not incident on the pupil when the pupil of the user moves (that is, when the viewpoint moves), and the user periodically observes an invisible region such as a black frame. Accordingly, it is difficult to say that sufficiently favorable display for the user is provided in the technology described in the above-described Patent Literature 1.

On the other hand, in the present embodiment, as described above, the size ds of the sampling region 207 is preferably 0.6 (mm) or less and a plurality of sampling regions 207 are set on the pupil as illustrated in FIG. 17. Then, light incident on each sampling region 207 is controlled. Accordingly, even when the viewpoint moves, there is no phenomenon in which pictures are discontinuously displayed as in the technology described in the above-described Patent Literature 1 and it is possible to provide the user with better display.

### (3-3-2. Iteration Cycle of Irradiation State of Sampling Region)

As described above, in the present embodiment, in order to cope with the movement of the user's viewpoint, a distance (DLP) between the lens surface 125 of the microlens array 120 and the pupil, a distance (that is, DXL) between the pixel array 110 and the microlens array 120, a pitch of the microlenses 121 in the microlens array 120, a pixel size and a pitch of the pixel array 110, and the like are set so that the irradiation state of light on each sampling region 207 is periodically iterated in units larger than the maximum pupil diameter of the user. The conditions required for the iteration cycle of the irradiation state of the sampling region 207 will be specifically examined.

The iteration cycle of the irradiation state of the sampling region 207 (hereinafter also simply referred to as an iteration cycle) can be set on the basis of the user's pupil distance (PD). Assuming that a group of sampling regions 207 corresponding to one cycle of iteration cycles is called a sampling region group for convenience, an iteration cycle λ corresponds to a size (length) of the sampling region group.

Normal viewing is hindered at the moment when the viewpoint of the user transits between sampling region groups. Accordingly, in order to decrease a frequency of occurrence of disturbance of such display in accordance with the movement of the viewpoint of the user, the optimum design of the iteration cycle λ is important.

For example, if the iteration cycle λ is larger than the PD, the left and right eyes can be included within the same iteration cycle. Accordingly, for example, the naked eye 3D display technology is used, so that it is possible to perform stereoscopic viewing as well as display for compensating for the visual acuity described in the above (3-2-2. Visual acuity compensation mode). Also, although normal viewing is hindered at the moment when the viewpoint of the user transits between the sampling region groups, the frequency of disturbance of such display can be decreased because the frequency of transition of the user's viewpoint between sampling region groups is lowered even when the viewpoint is moved by increasing the iteration cycle λ. In this manner, when implementing functions other than visual acuity compensation such as stereoscopic vision, it is preferable that the iteration cycle λ be as large as possible.

However, in order to increase the iteration cycle λ, it is necessary to increase the number of pixels 111 of the pixel array 110. An increase in the number of pixels causes manufacturing costs and power consumption to be increased. Accordingly, there is inevitably a limit to increasing the iteration cycle λ.

From the viewpoints of manufacturing costs and power consumption, when the iteration cycle λ is set to be equal to or less than PD, it is desirable that the iteration cycle λ be set to satisfy the following Equation (3). Here, n is an arbitrary natural number.

[Math. 3]

λ×n=PD (3)

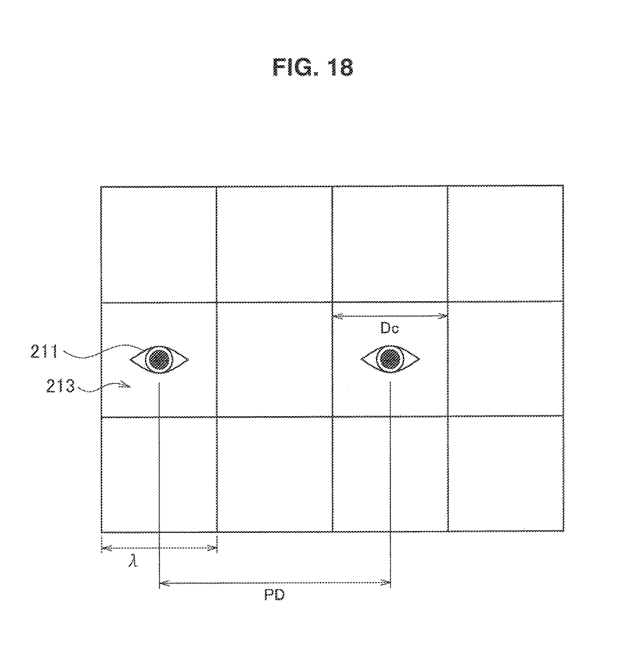

A relationship between λ and PD when the iteration cycle λ satisfies the above-described Equation (3) is illustrated in FIG. 18. FIG. 18 is a diagram illustrating the relationship between λ and PD when the iteration cycle λ satisfies Equation (3). Positional relationships between the sampling region group 213 including sampling regions 207 and the left and right eyes 211 of the user when the iteration cycle λ satisfies the above-described Equation (3) are illustrated in FIG. 18. In the example illustrated in FIG. 18, the sampling region group 213 is set as a substantially square region in a plane including the pupil of the user.

Here, as described above, normal viewing is hindered at the moment when the viewpoint of the user transits between the sampling region groups 213. However, when the iteration cycle λ satisfies the above-described Equation (3), for example, when the user's viewpoint moves in the left and right directions of the drawing sheet, the left and right eyes 211 pass through the boundary between the sampling region groups 213 at the same time. Accordingly, if a continuous region in which normal viewing is possible in both of the left and right eyes 211 is referred to as a continuous display region when the viewpoint moves, the continuous display region can be maximized when the iteration cycle λ satisfies the above-described Equation (3). In FIG. 18, a width Dc (continuous display width Dc) of the continuous display region in the left-right direction on the drawing sheet is indicated by a double-ended arrow. At this time, Dc=λ.

In contrast, when the iteration cycle λ is set to satisfy the following Equation (4), the continuous display region becomes the smallest.

[Math. 4]

λ×(n+0.5)=PD (4)

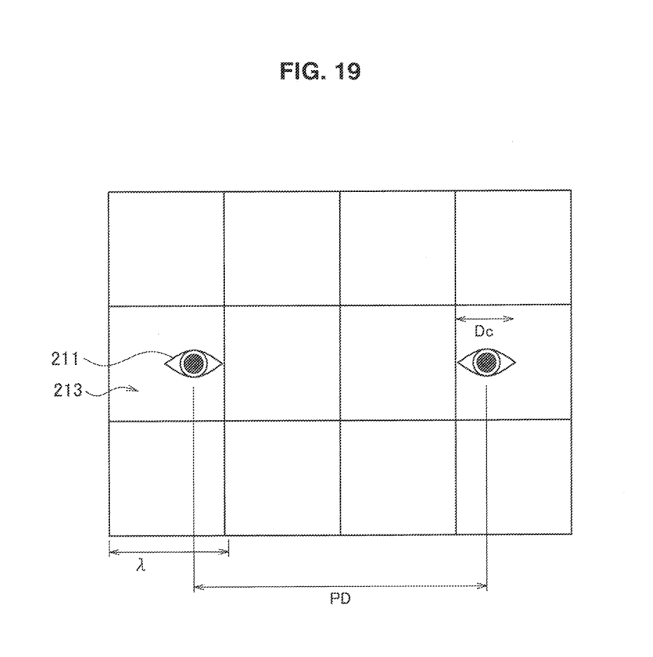

A relationship between λ and PD when the iteration cycle λ satisfies the above-described Equation (4) is illustrated in FIG. 19. FIG. 19 is a diagram illustrating the relationship between λ and PD when the iteration cycle λ satisfies Equation (4). Positional relationships between the sampling region group 213 including the sampling regions 207 and the left and right eyes 211 of the user when the iteration cycle λ satisfies Equation (4) are illustrated in FIG. 19.

In FIG. 19, as in FIG. 18, the width Dc (continuous display width Dc) in the left-right direction of the drawing sheet of the continuous display region is indicated by a double-end arrow. As illustrated in FIG. 19, when the iteration cycle λ satisfies the above-described Equation (4), if the left and right eyes 211 of the user only slightly move in the left-right direction of the drawing sheet, either one of the left and right eyes 211 will pass through the boundary between sampling region groups 213. Therefore, when the iteration cycle λ satisfies the above-described Equation (4), the continuous display region becomes smaller. At this time, Dc=λ/2.

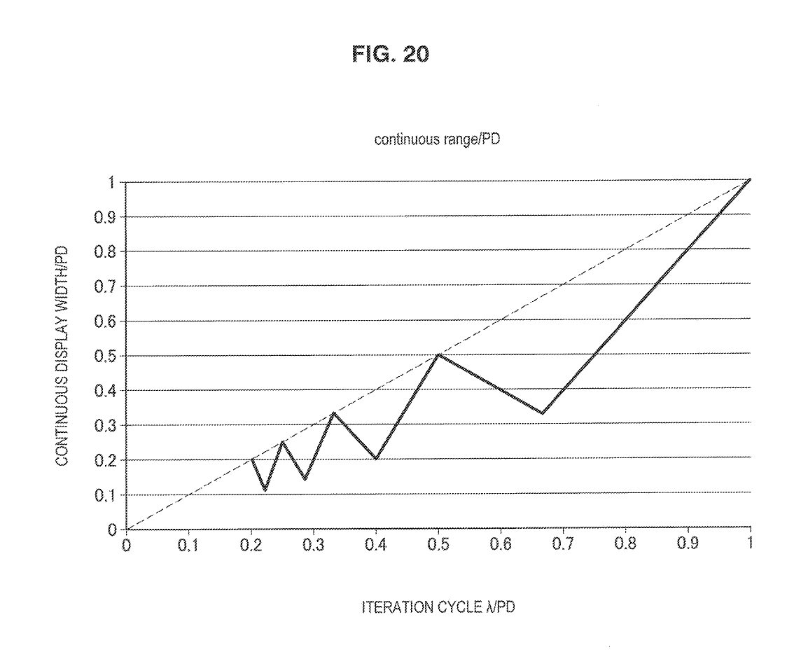

FIG. 20 is a diagram illustrating an influence of the relationship between the iteration cycle λ and the PD on the size of the continuous display region. In FIG. 20, a ratio between the iteration cycle λ and the PD (iteration cycle λ/PD) is taken on the horizontal axis, a ratio between the continuous display width Dc and PD (continuous display width Dc/PD) is taken on the vertical axis, and a relationship between the two ratios is plotted.

As illustrated in FIG. 20, when the iteration cycle λ satisfies the above-described Equation (3) (corresponding to the point where the value on the horizontal axis is 1, 1/2, 1/3, . . . ), the continuous display width Dc/PD has the same value as the iteration cycle λ/PD. That is, the continuous display width Dc takes λ which is a highest efficiency value.

On the other hand, when the iteration cycle λ satisfies the above-described Equation (4) (corresponding to the point where the value on the horizontal axis is 1/1.5, 1/2.5, 1/3.5, . . . ), the continuous display width Dc/PD takes a value of 1/2 of the iteration cycle λ/PD. That is, the continuous display width Dc takes λ/2 which is a lowest efficiency value.

The conditions required for the iteration cycle of the irradiation state of the sampling region 207 have been described above. As described above, it is also possible to apply the display device 10 to another field of application such as stereoscopic viewing by setting the iteration cycle λ of the irradiation state of the sampling region 207 to be larger than the PD. However, because it is necessary to increase the number of pixels 111 of the pixel array 110 in order to increase the iteration cycle λ, there is a limit in terms of manufacturing costs and power consumption. On the other hand, when an objective is to only compensate for the visual acuity, it is not always necessary to make the iteration cycle λ larger than PD. In this case, it is desirable that the iteration cycle λ be set to satisfy the above-described Equation (3). By setting the iteration cycle λ to satisfy the above-described Equation (3), the continuous display region can be maximized most efficiently and convenience for the user can be further improved.

### 4. Display Control Method

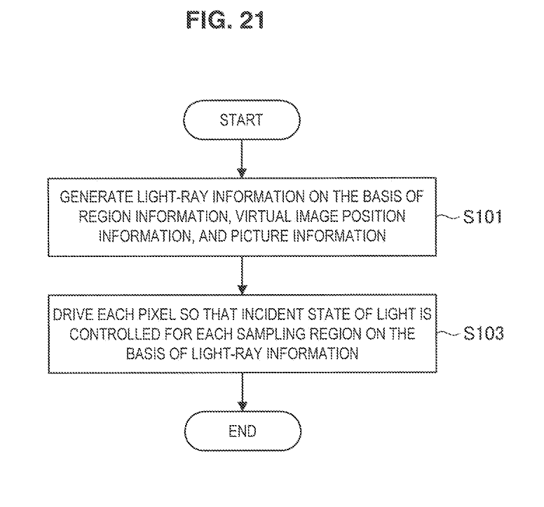

The display control method executed in the display device 10 according to the present embodiment will be described with reference to FIG. 21. FIG. 21 is a flowchart illustrating an example of a processing procedure of the display control method according to the present embodiment. Each process illustrated in FIG. 21 corresponds to that executed by the control unit 130 illustrated in FIG. 10.

Referring to FIG. 21, in the display control method according to the present embodiment, light-ray information is first generated on the basis of region information, virtual image position information, and picture information (step S101). The region information is information about a sampling region group including a plurality of sampling regions set on a plane including the user's pupil and substantially parallel to the display surface (the lens surface 125 of the microlens array 120) of the display device 10 illustrated in FIG. 10. Also, the virtual image position information is information about a position (virtual image generation position) at which a virtual image is generated in the display device 10 illustrated in FIG. 10. For example, the virtual image generation position is set to a position in focus for the user. Also, the picture information is two-dimensional picture information to be presented to the user.

In the process shown in step S101, information indicating the light-ray state is generated as light-ray information so that light from the picture based on the picture information displayed at the virtual image generation position based on the virtual image position information is incident on each sampling region included in the sampling region group. The light-ray information includes information about the emission state of light in each microlens 121 and information about the irradiation state of the light to each sampling region 207 for reproducing the light-ray state. Also, the process shown in step S101 corresponds to, for example, a process to be performed by the light-ray information generating unit 131 illustrated in FIG. 10.

Next, on the basis of the light-ray information, each pixel is driven so that the incident state of light is controlled for each sampling region (step S103). Thereby, the light-ray state as described above is reproduced, and a virtual image of a picture based on the picture information is displayed at the virtual image generation position based on the virtual image position information. That is, clear display in focus for the user is implemented.

The display control method according to the present embodiment has been described above.

### 5. Application Examples

Several application examples of the display device 10 according to the above-described present embodiment will be described.

### (5-1. Application to Wearable Device)

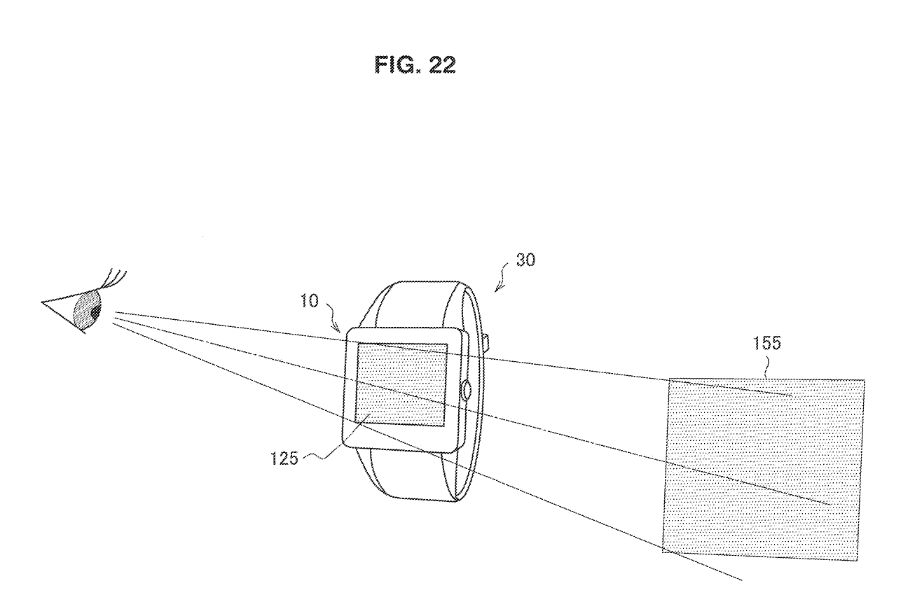

An example of a configuration in which the display device 10 according to the present embodiment is applied to a wearable device will be described with reference to FIG. 22. FIG. 22 is a diagram illustrating an example of a configuration in which the display device 10 according to the present embodiment is applied to a wearable device.

As illustrated in FIG. 22, the display device 10 according to the present embodiment can be preferably applied to a device having a relatively small display screen such as a wearable device 30. In the illustrated example, the wearable device 30 is a wristwatch type device.

In a mobile device such as the wearable device 30, the size of the display screen is limited to a relatively small size in consideration of portability for the user. However, as described in the above (1. Background of present disclosure), in recent years, the amount of information handled by users has increased and it is necessary to display more information on one screen. For example, there is a possibility that it will be difficult for a user with presbyopia to visually recognize the display on the screen due to simply increasing the amount of information displayed on the screen.

On the other hand, according to the present embodiment, as illustrated in FIG. 22, a virtual image 155 of a picture displayed on the display surface 125 can be generated at a position different from the real display surface 125. Accordingly, the user can observe fine display without wearing optical compensation instruments such as presbyopic glasses. Accordingly, even for a relatively small screen such as the wearable device 30, it is possible to perform high-density display and provide more information to the user.

### (5-2. Application to Other Mobile Device)

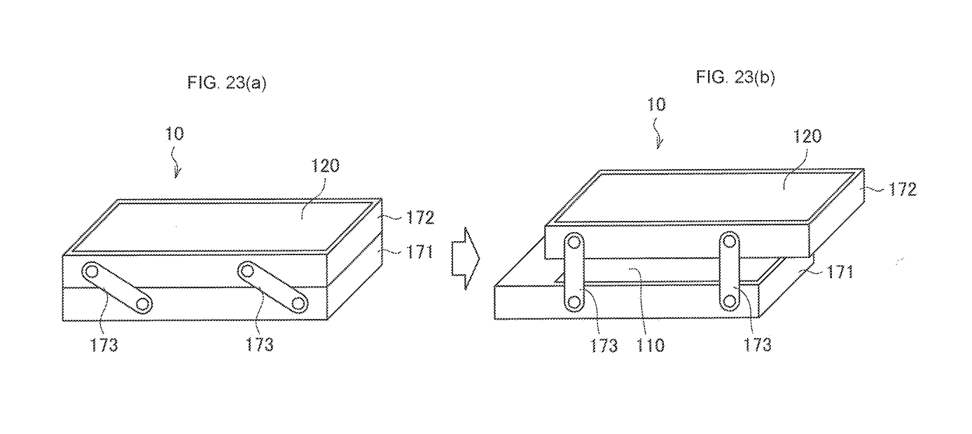

An example of a configuration in which the display device 10 according to the present embodiment is applied to another mobile device such as a smartphone will be described with reference to FIG. 23. FIG. 23 is a diagram illustrating an example of a configuration in which the display device 10 according to the present embodiment is applied to another mobile device.

In the example of the configuration illustrated in FIG. 23, when the display device 10 is mounted in a mobile device such as a smartphone, a first housing 171 on which the pixel array 110 is mounted and a second housing 172 on which the microlens array 120 is mounted are configured as different housings from each other and the first housing 171 and the second housing 172 are connected to each other by a connection member 173, so that the mobile device having the display device 10 is configured. The first housing 171 corresponds to the main body of the mobile device and a processing circuit for controlling the operation of the entire mobile device including the display device 10 and the like may be mounted within the first housing 171.

The connection member 173 is a bar-like member having rotary shaft portions provided at both ends thereof. As illustrated, one of the rotating shaft portions is connected to the side surface of the first housing 171 and the other of the rotating shaft portions is connected to the side surface of the second housing 172. In this manner, the first housing 171 and the second housing 172 are rotatably connected to each other by the connection member 173. Thereby, as illustrated, switching between a state in which the second housing 172 is in contact with the first housing 171 ((a) in FIG. 23) and a state in which the second housing 172 is located at a predetermined distance from the first housing 171 ((b) in FIG. 23) is performed.

Here, as described in the above (3-1. Device configuration), in the display device 10, the distance DXL between the lens surface 125 of the microlens array 120 and the display surface 115 of the pixel array 110 is an important factor for determining the projection size of the light beam on the pupil, the iteration cycle of the irradiation state of light with respect to each sampling region 207, and the like. However, if the mobile device is configured so that the predetermined DXL is always secured when the display device 10 is mounted on the mobile device, the volume of the mobile device is increased and the increase in the volume is not preferable from the viewpoint of portability. Accordingly, when mounting the display device 10 on the mobile device, it is preferable that a movable mechanism that makes the DXL variable be provided in the microlens array 120 and the pixel array 110.

The configuration illustrated in FIG. 23 shows an example of a configuration in which such a movable mechanism is provided in the display device 10. In the mobile device illustrated in FIG. 23, when the display device 10 is not used, the mobile device is set to a state in which the second housing 172 is in contact with the first housing 171 as illustrated in (a) of FIG. 23. In this state, the microlens array 120 and the pixel array 110 are arranged so that the DXL becomes smaller and the mobile device can be kept at a smaller volume. On the other hand, in the mobile device illustrated in FIG. 23, the length of the connection member 173 is adjusted so that the DXL becomes a predetermined distance taking into consideration the projection size of the light beam on the pupil and/or the iteration cycle of the irradiation state of light in the state in which the second housing 172 illustrated in (b) of FIG. 23 is located at a predetermined distance from the first housing 171. Accordingly, by setting the second housing 172 to be separated from the first housing 171 as illustrated in (b) of FIG. 23 when the display device 10 is used, it is possible to arrange the microlens array 120 and the pixel array 110 so that the DXL has a predetermined distance taking into consideration various conditions described above and perform display in the visual acuity compensation mode.

In this manner, by providing a mechanism for making the DXL variable when the display device 10 is mounted on a mobile device, both of the decrease of the volume when it is not used (that is, when it is carried) and the visual acuity compensation effect when it is used can coexist and convenience for the user can be further improved.

Also, even when the DXL is minimized when it is not used, the display device 10 can perform display in the normal mode. Because the lens effect in the microlens array 120 is also minimized when the DXL is minimized, display can be performed in the same manner as ordinarily (that is, there is no visual acuity compensation effect) due to the pixel array 110. Also, in the configuration example illustrated in FIG. 23, a movable mechanism that makes the distance between the first housing 171 and the second housing 172 variable is provided, but an example of a configuration of the mobile device is not limited to this example. For example, instead of or in addition to the movable mechanism, a detachable mechanism capable of detaching the second housing 172 from the first housing 171 may be provided. With an attaching/detaching mechanism, the mobile device can be kept at a small volume when the display device 10 is not used by detaching the second housing 172 from the first housing 171, and the second housing 172 is attached at a predetermined distance from the first housing 171 when the display device 10 is used and therefore display in the visual acuity compensation mode can be performed.

### (5-3. Application to Electronic Loupe Device)

Generally, a visual acuity compensation device (hereinafter referred to as an “electronic loupe device”) in which a camera is provided on the surface of a housing and information on the paper surface photographed by the camera is enlarged and displayed on a display screen provided on the back surface of the housing is known. A user can read an enlarged map, characters, or the like via the display screen by placing the electronic loupe device on, for example, a surface of paper such as a map or a newspaper, so that the camera faces the paper surface. The display device 10 according to the present embodiment can also be preferably applied to such an electronic loupe device.

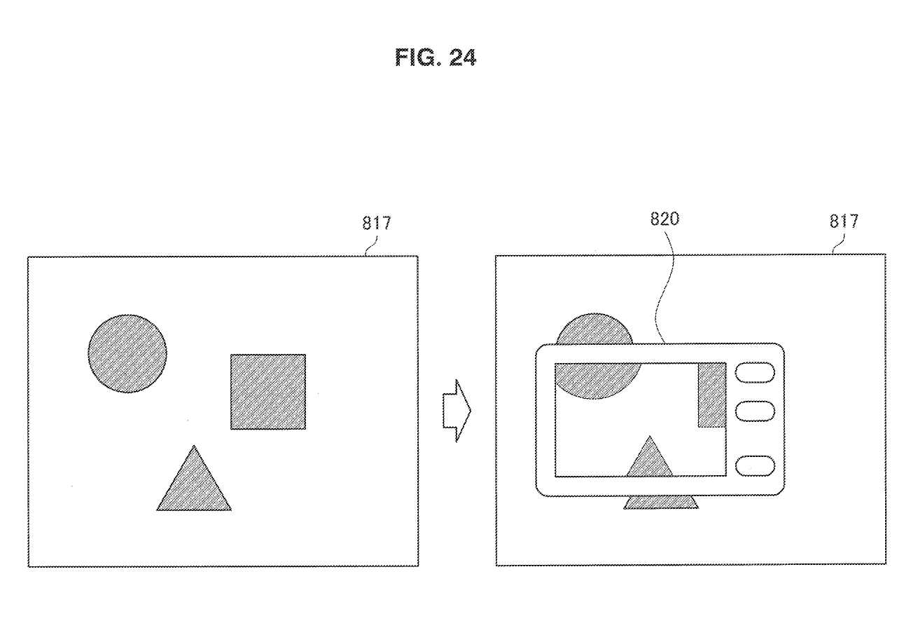

FIG. 24 illustrates an example of a general electronic loupe device. FIG. 24 is a diagram illustrating an example of a general electronic loupe device. As described above, the camera is mounted on the surface of the housing of the electronic loupe device 820. As illustrated, the electronic loupe device 820 is placed on a paper surface 817 so that the camera faces the paper surface 817. Graphics, characters, and the like on the paper surface 817 photographed by the camera are appropriately enlarged and displayed on the display screen of the back side of the housing of the electronic loupe device 820. Thereby, for example, a user who experiences difficulty in reading graphics and characters with small sizes due to presbyopia or the like can read the information on the paper surface more easily.

Here, the general electronic loupe device 820 as illustrated in FIG. 24 merely enlarges and displays a captured picture simply at a predetermined magnification, unlike a loupe made of optical lenses. Accordingly, because the user needs to enlarge the display to such an extent that it can be read without blur, the number of characters (an amount of information) to be displayed on the display screen at a time decreases. Consequently, when attempting to read a wide area of information within the paper surface 817, it is necessary to frequently move the electronic loupe device 820 on the paper surface 817.

On the other hand, when the display device 10 according to the present embodiment is mounted on the electronic loupe device, for example, a configuration example in which a camera is mounted on the front surface of the housing and the display device 10 is mounted on the back surface of the housing can be conceived. By placing the electronic loupe device so that the surface on which the camera is provided faces the paper surface and driving the electronic loupe device, a picture including information on the paper surface photographed by the camera can be displayed by the display device 10 mounted on the back surface of the housing.

If the display device 10 is driven in the visual acuity compensation mode, it is possible to perform display for remedying blur originally due to presbyopia or the like without enlarging the picture. As described above, in an electronic loupe device on which the display device 10 is mounted, unlike a general electronic loupe device 820, it is possible to perform visual acuity compensation without decreasing the amount of information to be displayed on the display screen at a time. Accordingly, even when a wide area of information within the paper surface is intended to be read, it is not necessary to frequently move the electronic loupe device on the paper surface and the user's readability can be significantly improved.

Several application examples of the display device 10 according to the present embodiment have been described above. However, the present embodiment is not limited to the above-described examples and the device to which the display device 10 is applied may be another device. For example, the display device 10 may be mounted on a mobile device in a form other than a wearable device or a smartphone. Alternatively, a device to which the display device 10 is applied is not limited to a mobile device and may be applied to any device as long as a device having a display function such as a stationary television is provided.

### 6. Modified Example

Several modified examples of the embodiment described above will be described.

(6-1. Decrease of Pixel Size in Accordance with Aperture)

As described in the above (3-1. Device configuration), in the display device 10, there are correlations between a projection size (corresponding to the sampling region) of light on the pupil from a pixel, image magnification, and a size (resolution) of a pixel 111 of the pixel array 110. Specifically, assuming that the size of the sampling region is ds, the size of the pixel 111 is dp, and the image magnification is m, they have a relationship shown in the following Equation (5).

[Math. 5]

ds=dp×m (5)

Also, the image magnification m is represented as a ratio between a viewing distance (a distance between the lens surface 125 of the microlens array 120 and the pupil illustrated in FIG. 10) DLP and a distance DXL between the lens surface 125 of the microlens array 120 and the display surface 115 of the pixel array 110 illustrated in FIG. 10 by the following Equation (6).

[Math. 6]

m=DLP/DXL (6)

Here, a focal length f of the microlens 121 is assumed to satisfy the following Equation (7).

[Math. 7]

1/f=1/DLP+1/DXL (7)

As shown in the above-described Equations (5) and (6), the size dp of the pixel 111 is determined by the image magnification of the projection system of the microlens 121 that projects the pixel 111 onto the user's pupil. For example, according to requirements of another design matter, when the DXL needs to be decreased in a product or when the DLP needs to be increased, the image magnification m may need to be increased and the size dp of the pixel 111 may need to be decreased.

Here, if the size dp of the pixel 111 is simply decreased, the number of pixels 111 included in the pixel array 110 is increased and the increase in the number of pixels 111 may be undesirable in terms of manufacturing costs or power consumption. Therefore, as a method of decreasing the size dp of the pixel 111 while keeping the size ds of the sampling region at a small value and without increasing the number of pixels, a method of decreasing the size dp of the pixel 111 using a shielding plate having an aperture may be conceived. Also, in order to distinguish it from a shielding plate provided with an aperture used in the following (2-5-2. Example of configuration of light emission point other than microlens), the shielding plate used to decrease the size dp of the pixel 111 may be referred to as a first shielding plate in the present description.

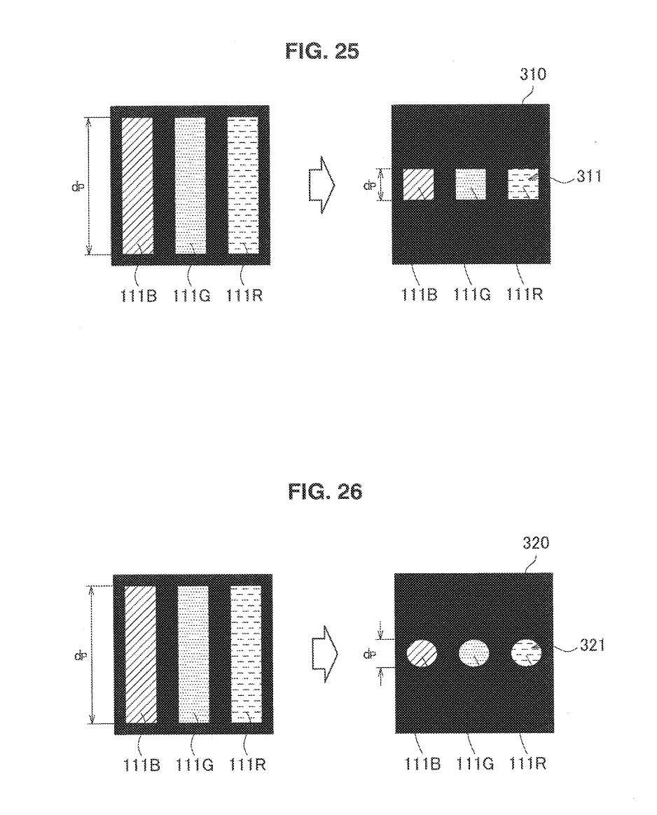

FIG. 25 is a schematic diagram illustrating a state of a decrease of a pixel size dp by a first shielding plate having a rectangular opening (aperture). Referring to FIG. 25, the shielding plate 310 is provided with a rectangular opening 311 at a position corresponding to each pixel 111 (111R, 111G, or 111B). A pixel 111R in FIG. 25 indicates a pixel that emits red light, a pixel 111G indicates a pixel that emits green light, and a pixel 111B indicates a pixel that emits blue light.

The size of the opening 311 is smaller than the sizes of the pixels 111R, 111G and 111B. By providing the shielding plate 310 to cover the pixels 111R, 111G and 111B, it is possible to apparently decrease the sizes dp of the pixels 111R, 111G and 111B.

FIG. 26 is a diagram illustrating an example of another configuration of the first shielding plate and is a schematic diagram illustrating a state of a decrease of a pixel size dp by a first shielding plate having a circular opening (aperture). Referring to FIG. 26, the shielding plate 320 is provided with a circular opening 321 at a position corresponding to each pixel 111 (111R, 111G or 111B). The size of the opening 321 is smaller than the sizes of the pixels 111R, 111G and 111B. By providing the shielding plate 320 to cover the pixels 111R, 111G and 111B, it is possible to apparently decrease the sizes dp of the pixels 111R, 111G and 111B.

Here, in the examples illustrated in FIGS. 25 and 26, the shielding plates 310 and 320 are provided on the display surface of the pixel array 110. However, in this modified example, the position at which the first shielding plate is provided is not limited to the display surface. For example, when the pixel array 110 is provided as a transmissive pixel array such as a pixel array of a liquid crystal display device, the first shielding plate may be provided between the backlight and the liquid crystal layer (liquid crystal panel) in the liquid crystal display device.

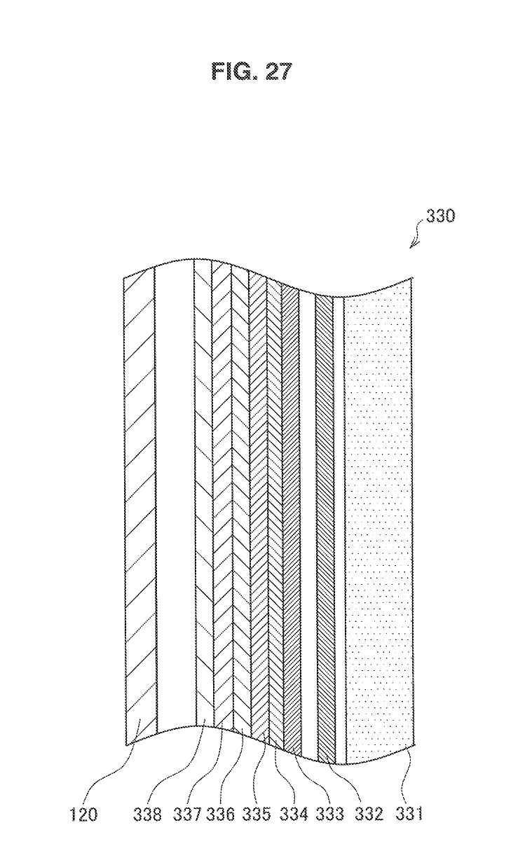

An example of a configuration in which such a first shielding plate is provided between the backlight and the liquid crystal layer is illustrated in FIG. 27. FIG. 27 is a diagram illustrating an example of a configuration in which the first shielding plate is provided between the backlight and the liquid crystal layer.

A cross-sectional view in a direction perpendicular to the display surface of a liquid crystal display device to which the first shielding plate is added is illustrated in FIG. 27. Referring to FIG. 27, the liquid crystal display device 330 includes a backlight 331, a diffusion plate 332, an aperture film 333, a polarization plate 334, a thin film transistor (TFT) substrate 335, a liquid crystal layer 336, a color filter substrate 337, and a polarization plate 338 stacked in this order. Because the configuration of the liquid crystal display device 330 is similar to that of a general liquid crystal display device except that the aperture film 333 is provided, a detailed description of the configuration will be omitted.

In this modified example, the pixel array of the liquid crystal display device 330 includes the pixel array 110 illustrated in FIG. 10. In FIG. 27, the microlens array 120 is also illustrated to correspond with FIG. 10.

The aperture film 333 corresponds to the above-described first shielding plates 310 and 320. The aperture film 333 has a configuration in which a plurality of optical openings (apertures (not illustrated)) are provided in correspondence with the positions of the pixels in the light shielding member and the light from the backlight 331 passes through the opening portion and is incident on the liquid crystal layer 336. Accordingly, because the aperture film 333 shields light outside a position at which the opening is provided, the pixel size is substantially decreased.

Here, a reflection layer that reflects light may be provided on the surface on the backlight side of the aperture film 333. When the reflection layer is provided, light from the backlight 331 that is not transmitted through the opening from light from the backlight 331 is reflected by the reflection layer toward the backlight 331. Reflected and returned light is reflected inside the backlight 331 again and emitted toward the aperture film 333 again. If there is no optical absorption in the reflecting surface of the aperture film 333 and the backlight 331, all the light is ideally reflected and incident on the liquid crystal layer 336 and loss of light is eliminated. Alternatively, a similar effect can be obtained also when the aperture film 333 itself of a material having high reflectance is formed instead of providing the reflection layer. In this manner, by providing a reflection layer on the surface of the aperture film 333 on the backlight side or by forming the aperture film 333 itself of a material with high reflectance, loss of light can be minimized even when the size of the opening is small, because light is recycled between the backlight 331 and the aperture film 333, so to speak.

Also, as another configuration, it is also possible to implement a configuration in which a positional relationship between the aperture film 333 and the liquid crystal layer 336 is reversed in the configuration example described above. In this case, it is possible to use a self-luminous type display device which is not a transmissive type instead of the liquid crystal layer 336.

A modified example in which the pixel size is decreased using the first shielding plate has been described above.

(6-2. Example of Configuration of Light Emission Point Other than Microlens)

In the above-described embodiment, the display device 10 is configured by arranging the microlens array 120 on the display surface of the pixel array 110. In the display device 10, each microlens 121 may function as a light emission point. Here, the present embodiment is not limited to such an example, and the light emission point may be implemented by a configuration other than a microlens.

For example, instead of the microlens array 120 illustrated in FIG. 10, a shielding plate having a plurality of openings (apertures) can be used. In this case, each opening of the shielding plate functions as a light emission point. Also, to distinguish it from the shielding plate used in the above (6-1. Decrease of pixel size in accordance with aperture), a shielding plate used for configuring a light emission point instead of the microlens array 120 may be referred to as a second shielding plate in the present description.

The second shielding plate may have a configuration substantially similar to a parallax barrier used for a general 3D display device. In this modified example, a shielding plate having an opening at a position corresponding to the center of each microlens 121 illustrated in FIG. 10 is arranged on the display surface 115 of the pixel array 110 instead of the microlens array 120.

From optical considerations similar to the above-described Equations (5) and (6), the projection size of light (which corresponds to the sampling region) becomes ((pixel size of pixel array 110)+(diameter of aperture))×(distance between shielding plate and pupil)/(distance between pixel array 110 and shielding plate) when light from the pixel 111 passes through the opening of the shielding plate and is projected onto the pupil of the user. Accordingly, in consideration of the size of the sampling region of 0.6 (mm) or less, the opening of the shielding plate can be designed to satisfy the above-described conditions.

Here, when a shielding plate is used instead of the microlens array 120, light not passing through the opening is not emitted toward the user, resulting in a loss. Accordingly, compared with when the microlens array 120 is provided, the display observed by the user may become dark. Accordingly, when a shielding plate is used instead of the microlens array 120, it is preferable that each pixel be driven in consideration of such loss of light.

Also, when the pixel array 110 is configured using a transmissive display device such as a liquid crystal display device, a configuration in which the positional relationship between the second shielding plate and the transmissive pixel array 110 is reversed can also be similarly implemented. In this case, for example, the second shielding plate is arranged between the backlight and the liquid crystal layer. In this case, as in the configuration described above with reference to FIG. 27, it is possible to obtain the effect of decreasing light loss by providing a reflection layer on the backlight side surface of the second shielding plate or forming the second shielding plate itself with a material having high reflectance.

A modified example in which the light emission point is implemented by a configuration other than a microlens has been described above.

(6-3. Dynamic Control of Irradiation State in Accordance with Pupil Position Detection)

As described in the above (3-1. Device configuration), the display device 10 according to the present embodiment sets a sampling region group including a plurality of sampling regions on a plane including the user's pupil and controls the irradiation state of light for each sampling region. Also, as described in the above (3-3-2. Iteration cycle of irradiation state of sampling region), the irradiation state of light for each sampling region is iterated in a predetermined cycle. Here, when the user's eyes pass through a boundary between the sampling region groups corresponding to one cycle of iteration, the user does not recognize normal display.

As one method of avoiding such abnormal display when the viewpoint passes through the boundary between the sampling region groups, it is conceivable to increase the iteration cycle λ of the irradiation state of the sampling region. However, as described in the above (3-3-2. Iteration cycle of irradiation state of sampling region), when the iteration cycle λ is increased, the number of pixels in the pixel array is increased, the pixel pitch is decreased, power consumption is increased, and the like, thereby causing problems in terms of product specifications.

Therefore, as another method of avoiding abnormal display when the viewpoint passes through the boundary between the sampling region groups, a method of detecting a position of the user's pupil and dynamically controlling the irradiation state of the sampling region in accordance with the detected position may be conceived.

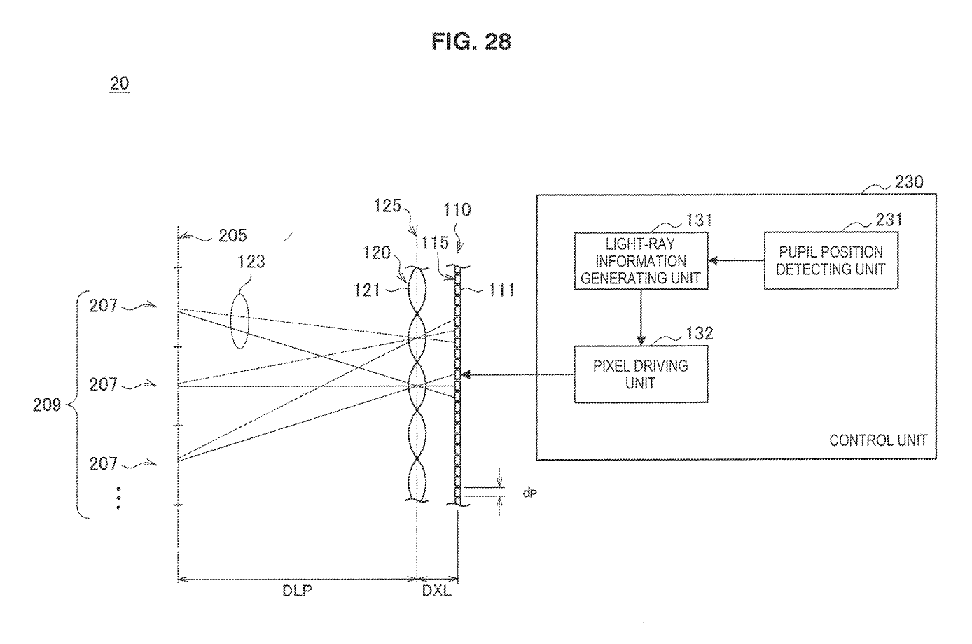

A configuration of a display device for implementing such dynamic control of the irradiation state in accordance with pupil position detection will be described with reference to FIG. 28. FIG. 28 is a diagram illustrating an example of a configuration of a display device according to a modified example in which dynamic control of the irradiation state in accordance with the pupil position detection is performed.

Referring to FIG. 28, the display device 20 according to the present modified example includes a pixel array 110 in which a plurality of pixels 111 are two-dimensionally arranged, a microlens array 120 provided on a display surface 115 of the pixel array 110, and a control unit 230 that controls driving of each pixel 111 of the pixel array 110. Each pixel 111 is driven by the control unit 230 on the basis of the light-ray information, so that, for example, the light-ray state of light from a picture on a virtual image surface located at a predetermined position is reproduced. Here, because the configurations and functions of the pixel array 110 and the microlens array 120 are similar to the configurations and functions of these members in the display device 10 illustrated in FIG. 10, a detailed description thereof will be omitted here.

The control unit 230 includes, a processor such as a CPU or a DSP, and operates in accordance with a predetermined program, thereby controlling the driving of each pixel 111 of the pixel array 110. The control unit 230 has a light-ray information generating unit 131, a pixel driving unit 132, and a pupil position detecting unit 231 as functions thereof. Because the functions of the light-ray information generating unit 131 and the pixel driving unit 132 are substantially similar to the functions of these configurations in the display device 10 illustrated in FIG. 10, description of matters repeated from the control unit 130 of the display device 10 will be omitted and differences from the control unit 130 will mainly be described here.

On the basis of the region information, the virtual image position information and the picture information, the light-ray information generating unit 131 generates information indicating the light-ray state when light from a picture displayed on the virtual image surface is incident on each sampling region 207 as light-ray information. For example, the information about the cycle (iteration cycle λ) of iteratively reproducing the irradiation state of light for each sampling region 207 may be included in the region information. When the light-ray information is generated, the light-ray information generating unit 131 generates information about the irradiation state of light for each sampling region 207 in consideration of the iteration cycle λ.

The pixel driving unit 132 drives each pixel 111 of the pixel array 110 so that the incident state of light is controlled for each sampling region 207 on the basis of the light-ray information. Thereby, the above-described light-ray state is reproduced and a virtual image is displayed to the user.

The pupil position detecting unit 231 detects the position of the user's pupil. As a method in which the pupil position detecting unit 231 detects the position of the pupil, for example, any known method used in general visual line detection and the like may be applied. For example, an imaging device (not illustrated) capable of photographing at least the face of the user may be provided in the display device 20, and the pupil position detecting unit 231 analyzes a captured picture acquired by the imaging device using a well-known picture analysis method, thereby detecting the position of the user's pupil. The pupil position detecting unit 231 provides information about the detected pupil position of the user to the light-ray information generating unit 131.

In the present modified example, the light-ray information generating unit 131 generates information about the irradiation state of light for each sampling region 207 so that that the pupil of the user is not positioned at a boundary between the sampling region groups, which are units of iterations of the irradiation state for each sampling region 207, on the basis of information about the position of the pupil of the user. The light-ray information generating unit 131 generates information about the irradiation state of light for each sampling region 207, for example, so that the user's pupil is always located at substantially the center of a sampling region group.

Each pixel 111 is driven by the pixel driving unit 132 on the basis of the above-described light-ray information, so that the position of the sampling region group in the sampling region groups 209 may be changed at any time in accordance with the movement of the position of the user's pupil in the present modified example so that the pupil is not positioned at a boundary between the sampling region groups. Accordingly, it is possible to prevent the viewpoint of the user from passing through a boundary between sampling region groups and it is possible to avoid the occurrence of abnormal display when the user's viewpoint passes through a boundary. Consequently, it is possible to decrease the stress of the user using the display device 20. Also, according to the present modified example, as in the case in which the iteration cycle λ is increased, the manufacturing costs and the power consumption are not increased, so that more comfortable display and optimization of costs, etc. can be compatible.

A modified example in which dynamic control of the irradiation state is performed in accordance with pupil position detection has been described above.

(6-4. Modified Example in which Pixel Array is Implemented by Printing Material)

Although the pixel array 110 is implemented as a configuration of a display device such as, for example, a liquid crystal display device, in the display device 10 described in the above (3-1. Device configuration), the present embodiment is not limited to such an example. For example, the pixel array 110 may be implemented by a printing material.

When the pixel array 110 is implemented by a printing material in the display device 10 illustrated in FIG. 10, a printing control unit can be provided instead of the pixel driving unit 132 as a function of the control unit 130. The printing control unit has a function of obtaining information to be displayed on the printing material through calculation on the basis of the light-ray information generated by the light-ray information generating unit 131 and controlling the operation of a printing unit including a printing device such as a printer so that information similar to that when the information is displayed on the pixel array 110 is printed on the printing material. The printing unit may be incorporated in the display device 10 or may be provided as a separate device different from the display device 10.

By arranging the printing material printed under the control of the printing control unit at the position of the pixel array 110 illustrated in FIG. 10 instead of the pixel array 110 and by using appropriate illumination as necessary, it is possible to display a virtual image at a predetermined position to the user and perform display for compensating for the visual acuity of the user as in the display device 10.

### 7. Supplement

The preferred embodiment(s) of the present disclosure has/have been described above with reference to the accompanying drawings, whilst the present disclosure is not limited to the above examples. A person skilled in the art may find various alterations and modifications within the scope of the appended claims, and it should be understood that they will naturally come under the technical scope of the present disclosure.

Further, the effects described in this specification are merely illustrative or exemplified effects, and are not limitative. That is, with or in the place of the above effects, the technology according to the present disclosure may achieve other effects that are clear to those skilled in the art from the description of this specification.

Also, the device configuration of the display device 10 according to the present embodiment is not limited to the example illustrated in FIG. 10. Further, the device configuration of the display device 20 according to the present modified example is not limited to the example illustrated in FIG. 28. For example, the functions of the control unit 130 may not necessarily be integrally mounted in one device. The functions of the control unit 130 may be distributed and mounted on a plurality of devices (for example, a plurality of processors) and the plurality of devices may be connected to communicate with one another so that the functions of the above-described control unit 130 may be implemented. Similarly, the functions of the control unit 230 may not necessarily be integrally mounted in one device. The functions of the control unit 230 may be distributed and mounted on a plurality of devices (for example, a plurality of processors) and the plurality of devices may be connected to communicate with one another so that the functions of the above-described control unit 230 may be implemented.

Also, a computer program for implementing the functions of the control unit 130 of the display device 10 according to the present embodiment and/or the control unit 230 of the display device 20 according to the present modified example as described above can be manufactured and mounted on a personal computer or the like. Also, it is possible to provide a computer-readable recording medium in which such a computer program is stored. The recording medium is, for example, a magnetic disk, an optical disc, a magneto-optical disc, a flash memory, or the like. Also, the computer program may be distributed via, for example, a network, without using a recording medium.

Additionally, the present technology may also be configured as below.

(1)

A display device including:

a plurality of light emission points, wherein

a region group including a plurality of regions which are set on a plane including a pupil of a user is irradiated with light emitted from each of the plurality of light emission points,

each of the plurality of light emission points causes light corresponding to a combination of the light emission point and the region to be incident on each of the regions, and

a number of the regions set on the pupil of the user is two or more, and a size of each of the regions is smaller than 0.6 (mm).

(2)

The display device according to (1), wherein

each of the plurality of light emission points causes the light corresponding to the combination of the light emission point and the region to be incident on each of the regions so that light from an image on a virtual display surface is formed on a retina of the user, the virtual display surface being different from a display surface including the plurality of light emission points.

(3)

The display device according to (2), wherein

the virtual display surface is located farther away from the user than the display surface including the plurality of light emission points.

(4)

The display device according to any one of (1) to (3), wherein

an irradiation state of the light with respect to each of the regions is periodically iterated in units larger than a maximum pupil diameter of the user.

(5)

The display device according to (4), wherein

an iteration cycle of the region group is larger than a pupil distance of the user.

(6)

The display device according to (4), wherein

a value obtained by multiplying an iteration cycle of the region group by an integer is substantially equal to a pupil distance of the user.

(7)

The display device according to any one of (1) to (6), further including:

a pixel array in which a plurality of pixels are arranged; and

a microlens array provided on a display surface of the pixel array, wherein

light from the plurality of pixels is emitted from each of microlenses included in the microlens array, so that each of the microlenses constitutes each of the light emission points, and

a pitch of the microlenses in the microlens array is larger than a pitch of the pixels in the pixel array.

(8)

The display device according to (7), wherein

a first shielding plate having a plurality of openings corresponding to the respective pixels in the pixel array is provided on the display surface of the pixel array, so that a size of each of the pixels is decreased by each of the openings.

(9)

The display device according to (7), wherein

the pixel array is a transmissive pixel array, and

a first shielding plate having a plurality of openings corresponding to the respective pixels in the pixel array is provided on a light source side of the pixel array, so that a size of each of the pixels is decreased by each of the openings.

(10)

The display device according to any one of (1) to (6), further including:

a pixel array in which a plurality of pixels are arranged; and

a second shielding plate provided on a display surface of the pixel array and having a plurality of openings, wherein

light from the plurality of pixels is emitted from each of the openings of the second shielding plate, so that each of the openings constitutes each of the light emission points, and

a pitch of the openings in the second shielding plate is larger than a pitch of the pixels in the pixel array.

(11)

The display device according to any one of (1) to (6), further including:

a transmissive pixel array in which a plurality of pixels are arranged; and

a second shielding plate provided on a light source side of the pixel array and having a plurality of openings, wherein

light from the openings of the second shielding plate is emitted from each of the plurality of pixels, so that each of the openings constitutes each of the light emission points, and

a pitch of the openings in the second shielding plate is larger than a pitch of the pixels in the pixel array.

(12)

The display device according to any one of (1) to (6), further including:

a pixel array in which a plurality of pixels are arranged, the pixel array being implemented by printing; and

a microlens array provided on a display surface of the pixel array, wherein

light from the plurality of pixels is emitted from each of microlenses included in the microlens array, so that each of the microlenses constitutes each of the light emission points, and

a pitch of the microlenses in the microlens array is larger than a pitch of the pixels in the pixel array.

(13)

The display device according to any one of (1) to (12), wherein

an irradiation state of the light with respect to each of the regions is periodically iterated in units larger than a maximum pupil diameter of the user, and

the irradiation state of the light to be incident on each of the regions from each of the plurality of light emission points is controlled so that the pupil of the user is not positioned at a boundary between iterations of the irradiation state of the light in accordance with a position of the pupil of the user.

(14)

A display control method including:

irradiating a region group including a plurality of regions which are set on a plane including a pupil of a user with light emitted from each of a plurality of light emission points, and also causing light corresponding to a combination of the light emission point and the region to be incident on each of the regions from each of the plurality of light emission points, wherein a number of the regions set on the pupil of the user is two or more, and a size of each of the regions is smaller than 0.6 (mm).

### REFERENCE SIGNS LIST

10, 20 display device
30 wearable device
110 pixel array
111 pixel
120 microlens array
121 microlens
130, 230 control unit
131 light-ray information generating unit
132 pixel driving unit
150 virtual image surface
231 pupil position detecting unit
310, 320, 333 first shielding plate (aperture film)
311, 321 opening

1. A display device comprising:
a plurality of light emission points, wherein
a region group including a plurality of regions which are set on a plane including a pupil of a user is irradiated with light emitted from each of the plurality of light emission points,
each of the plurality of light emission points causes light corresponding to a combination of the light emission point and the region to be incident on each of the regions, and
a number of the regions set on the pupil of the user is two or more, and a size of each of the regions is smaller than 0.6 (mm).

2. The display device according to claim 1, wherein
each of the plurality of light emission points causes the light corresponding to the combination of the light emission point and the region to be incident on each of the regions so that light from an image on a virtual display surface is formed on a retina of the user, the virtual display surface being different from a display surface including the plurality of light emission points.

3. The display device according to claim 2, wherein
the virtual display surface is located farther away from the user than the display surface including the plurality of light emission points.

4. The display device according to claim 1, wherein
an irradiation state of the light with respect to each of the regions is periodically iterated in units larger than a maximum pupil diameter of the user.

5. The display device according to claim 4, wherein
an iteration cycle of the region group is larger than a pupil distance of the user.

6. The display device according to claim 4, wherein
a value obtained by multiplying an iteration cycle of the region group by an integer is substantially equal to a pupil distance of the user.

7. The display device according to claim 1, further comprising:
a pixel array in which a plurality of pixels are arranged; and
a microlens array provided on a display surface of the pixel array, wherein
light from the plurality of pixels is emitted from each of microlenses included in the microlens array, so that each of the microlenses constitutes each of the light emission points, and
a pitch of the microlenses in the microlens array is larger than a pitch of the pixels in the pixel array.

8. The display device according to claim 7, wherein
a first shielding plate having a plurality of openings corresponding to the respective pixels in the pixel array is provided on the display surface of the pixel array, so that a size of each of the pixels is decreased by each of the openings.

9. The display device according to claim 7, wherein
the pixel array is a transmissive pixel array, and
a first shielding plate having a plurality of openings corresponding to the respective pixels in the pixel array is provided on a light source side of the pixel array, so that a size of each of the pixels is decreased by each of the openings.

10. The display device according to claim 1, further comprising:
a pixel array in which a plurality of pixels are arranged; and
a second shielding plate provided on a display surface of the pixel array and having a plurality of openings, wherein
light from the plurality of pixels is emitted from each of the openings of the second shielding plate, so that each of the openings constitutes each of the light emission points, and
a pitch of the openings in the second shielding plate is larger than a pitch of the pixels in the pixel array.

11. The display device according to claim 1, further comprising:
a transmissive pixel array in which a plurality of pixels are arranged; and
a second shielding plate provided on a light source side of the pixel array and having a plurality of openings, wherein
light from the openings of the second shielding plate is emitted from each of the plurality of pixels, so that each of the openings constitutes each of the light emission points, and
a pitch of the openings in the second shielding plate is larger than a pitch of the pixels in the pixel array.

12. The display device according to claim 1, further comprising:
a pixel array in which a plurality of pixels are arranged, the pixel array being implemented by printing; and
a microlens array provided on a display surface of the pixel array, wherein
light from the plurality of pixels is emitted from each of microlenses included in the microlens array, so that each of the microlenses constitutes each of the light emission points, and
a pitch of the microlenses in the microlens array is larger than a pitch of the pixels in the pixel array.

13. The display device according to claim 1, wherein
an irradiation state of the light with respect to each of the regions is periodically iterated in units larger than a maximum pupil diameter of the user, and
the irradiation state of the light to be incident on each of the regions from each of the plurality of light emission points is controlled so that the pupil of the user is not positioned at a boundary between iterations of the irradiation state of the light in accordance with a position of the pupil of the user.

14. A display control method comprising:
irradiating a region group including a plurality of regions which are set on a plane including a pupil of a user with light emitted from each of a plurality of light emission points, and also causing light corresponding to a combination of the light emission point and the region to be incident on each of the regions from each of the plurality of light emission points, wherein
a number of the regions set on the pupil of the user is two or more, and a size of each of the regions is smaller than 0.6 (mm).
